(19) United States
(12) Patent Application Publication
(10) Pub. No.: US 2016/0175238 A1
(43) Pub. Date: Jun. 23, 2016

(54) HAIR COSMETIC COMPOSITION CONTAINING A POLYURETHANE LATEX POLYMER AND A SILICONE-ORGANIC POLYMER COMPOUND

(71) Applicant: L'OREAL, Paris (FR)

(72) Inventors: Christine SHIN, East Brunswick, NJ (US); Allison BLACK, Metuchen, NJ (US)

(21) Appl. No.: 14/578,122

(22) Filed: Dec. 19, 2014

(51) Int. Cl. A61K 8/895; A61Q 17/04; A45D 7/06; A61Q 5/06; A61K 8/34; A61K 8/87; A61K 8/04

(52) U.S. Cl. CPC A61K 8/895; A61K 8/87; A61Q 17/04; A61K 8/046; A61Q 5/06; A61K 8/342; A61K 8/345; A45D 7/06

(57) ABSTRACT

Disclosed are hair cosmetic compositions comprising, in a cosmetically acceptable carrier, at least one polyurethane latex polymer and at least one silicone-organic polymer compound. Methods of imparting durable styling or shaping benefits to hair and/or of controlling frizziness of hair by applying the hair cosmetic compositions onto hair are also disclosed.

### FIELD OF THE INVENTION

The present application relates to a shampoo- and/or wash-resistant hair cosmetic composition containing a latex polymer, a silicone-organic polymer compound, an organic solvent, and water, and to a method of imparting durable styling or shaping properties to hair and/or controlling the frizziness of hair using the hair cosmetic composition of the present invention.

### BACKGROUND OF THE INVENTION

Consumers of hair cosmetic products such as hair care and hair styling products actively seek out multi-functional, new products that can impart good styling benefits to hair, are durable or last long on the hair, even with several washings or shampooings and can impart other cosmetic attributes. At the same time, such products have to be pleasing to the senses, both on application and in use, and which have innovative, interesting and/or pleasing textures, preferably without any sacrifice to functional performance. In addition, many consumers seek hair care products which provide a light feel, are easy to apply, moisturize or condition, and add shine to the hair. The resulting feel and texture of the product during the application process, in addition to the feel of the hair after the application, are also important elements of such commodities.

It is also highly desirable for manufacturers of hair cosmetic products to make products that prevent or minimize the frizziness of hair, particularly under high humidity conditions in which hair tends to absorb moisture causing it to be less manageable, which makes it more difficult to shape and style hair. Applying a coating, such as a moisture barrier or a film on the hair can help to keep moisture out of the hair, allowing for more efficient hair shaping and maintenance of hair shape, even in extreme humidity conditions.

Thus one challenge for manufacturers of hair cosmetic products is in making products that help hair maintain its shape or style even after several washings or shampooings/washings. These products are also expected to impart long lasting or durable frizz control benefits to hair, especially under very humid conditions.

Traditional compositions on the cosmetic market appear in various forms. They can range anywhere from solutions, foams, gels, creams, waxes, mousses, sprays, serums, to aerosols and can impart a variety of levels of care and cosmeticity depending on the state of the hair and skin. However, these conventional cosmetic compositions contain emulsifying systems which may have limitations and may be less appealing to the consumer. For example, the use of silicone compounds in some of these compositions to achieve desirable shine or certain textures and feel may result in other limitations. Such limitations may include sticky or greasy products, irritation on the skin/scalp, a heavy or oily feel to the hair and skin, and the use of high levels of raw materials or additional ingredients to correct for the detrimental effects of other ingredients, leading to a costly product. Therefore, there is still a need to improve currently marketed commodities in order to provide the consumer with innovative formulations that present sensory, functionality and cost-effective perspectives on cosmetic products.

Other drawbacks associated with current products for styling or shaping the hair include that the product is often sticky or tacky and/or often produces a film that imparts a sticky or tacky feel, and styled hair that is stiff and/or “crunchy” (i.e. the film is hard and brittle resulting in a crunching feel or sound when the hair is touched), which is undesirable for most consumers.

Current products for styling or shaping the hair typically include water soluble film-forming polymers. Depending on the chemical make-up of these polymers, they may be either soluble in water, or they may be water insoluble polymers which are made water soluble via various chemical modifications, such as neutralization. Solutions comprising these polymers tend to be viscous, i.e. as the concentration of the polymer increases, its viscosity builds up rapidly. Translated to styling applications, as the solvent evaporates, the polymer solution becomes thicker on the hair surface, resulting in a sticky or tacky film. These products also tend to exhibit problems with product spreadability, hair manageability, and low degree of humidity resistance which is particularly a problem in hot and humid countries.

Thus, the ability to maintain the shape of hair, and achieve frizz control, styling hold, good texture and shine on hair, while providing a clean, natural and light-weight feel to the hair remain as additional areas for improvement, particularly in connection with certain type of polymers such as silicone-based polymers.

It has now been surprisingly and unexpectedly discovered that by providing a composition comprising at least one polyurethane latex polymer, at least one silicone-organic compound, at least one organic solvent and water, it is possible to form a film on a substrate and impart certain desirable properties, such as durable or long lasting style and frizz control to hair, and in some instances, even after shampooing and/or washing the hair. It has also been surprisingly discovered that at certain ratios of the latex polymer to the silicone-organic compound, said composition can impart shape and/or maintain the shape of hair, provide frizz control benefits under high humidity conditions and a wash resistant style, a good styling hold, and good texture and shine to the hair as well as a clean, natural, and/or “invisible” feel, and a lack of stickiness. Such compositions may be useful in hair-styling applications wherein styling benefits such as a natural look, curling or straightening, and frizz control are desired.

Moreover, compositions according to embodiments of the invention may be prepared that can deliver a broad range of hair styling benefits, such as, for example, from low to high style-hold and hair straightening properties, for example by varying the weight ratio between the latex polymer and the silicone organic polymer compound, with or without additives.

### SUMMARY OF THE INVENTION

The present invention is directed to a hair cosmetic composition containing, in a cosmetically acceptable carrier:
(a) from about 0.25% to about 3.5% by weight of at least one polyurethane latex polymer; and
(b) from about 0.5% to about 6% by weight of at least one silicone-organic polymer compound;
   all weights being relative to the weight of the composition, and wherein the weight ratio of the polyurethane latex polymer to the silicone-organic polymer compound is less than 2.

The present invention also relates to a method of controlling frizziness of hair, comprising applying the above-described composition to the hair.

The present invention further relates to a method of styling or shaping hair or maintaining the shape of hair, comprising applying the above-described composition to the hair. Such styling methods may comprise shaping, reshaping, positioning, repositioning, adding volume to, curling, or straightening the hair, in order to achieve a certain hair style or appearance.

It has been surprisingly and unexpectedly discovered that the application of the composition of the present invention onto hair resulted in desirable and beneficial effects on the hair, for example, frizz-control, styling and shaping effects on hair, in some instances, even after shampooing and/or washing the hair, manageability of hair, humidity resistance, and hair straightening. Additional advantages can be achieved such as smoothness, softness, and clean/natural and light-weight/non-greasy or non-oily feel as well as no flaking of the composition on hair or other surfaces.

The compositions of the present invention can be formulated as a spray product having a low content of volatile organic solvents/compounds.

### DETAILED DESCRIPTION

As used herein, the expression “at least one” means one or more and thus includes individual components as well as mixtures/combinations.

It is also to be understood that, as used herein the terms “the,” “a,” or “an,” mean “at least one,” are understood to encompass the plural as well as the singular and should not be limited to “only one” unless explicitly indicated to the contrary. Thus, for example, the use of “an acid” is intended to mean at least one acid.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The term “comprising” (and its grammatical variations) as used herein is used in the inclusive sense of “having” or “including” and not in the exclusive sense of “consisting only of”.

“Keratin fiber” as used herein, includes, but is not limited to hair, such as hair on the human head and eyelashes.

“Film former” or “film forming polymer” as used herein means a polymer or resin that leaves a film on the substrate to which it is applied, for example, after a solvent accompanying the film former has evaporated, absorbed into and/or dissipated on the substrate. These terms may also refer to a polymer capable, by itself or in the presence of an auxiliary film forming polymer, of forming a continuous or a discontinuous film that adheres to a support and especially to keratin substrates such as keratin fibers or hair.

“Film former” or “film forming polymer” as used herein may also be referred to as fixing polymers when such polymers are employed to fix or keep keratin fibers in a particular configuration or shape or arrangement.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The terms “organic compound” and “having an organic structure” mean compounds containing carbon atoms and hydrogen atoms and optionally heteroatoms such as S, O, N or P, alone or in combination.

“Volatile”, as used herein, means having a flash point of less than 100° C.

“Non-volatile”, as used herein, means having a flash point of equal to or greater than 100° C.

As used herein, “formed from,” means obtained from chemical reaction of, wherein “chemical reaction,” includes spontaneous chemical reactions and induced chemical reactions. As used herein, the phrase “formed from,” is open ended and does not limit the components of the composition to those listed.

The term “stable” as used herein means that the composition does not exhibit phase separation and/or crystallization.

As used herein, the terms “applying a composition onto keratin fibers” and “applying a composition onto hair” and variations of these phrases are intended to mean contacting the fibers or hair, with at least one of the compositions of the invention, in any manner.

The term “treat” (and its grammatical variations) as used herein refers to the application of the compositions of the present invention onto keratin fibers such as hair.

The term “style” or styling” as used herein includes shaping, straightening, curling, or placing a keratin fiber such as hair, in a particular arrangement, form or configuration; or altering the curvature of a keratin fiber or other substrate; or re-positioning a keratin fiber or other substrate to a different arrangement, form or configuration.

As used herein, the terms “method of styling keratin fibers” or “method of styling hair” is understood to mean any method for modifying the appearance of the keratin fibers or the hair with respect to their spatial arrangement or configuration or curvature or form. When the keratin fibers comprise hair on the human head, the term “method of styling keratin fibers” or “method of styling hair” is also understood to mean any method for curling or waving or embossing the hair or smoothing or straightening the hair, or spiking the hair.

The compositions and methods of the present invention can comprise, consist of, or consist essentially of the essential elements and limitations of the invention described herein, as well as any additional or optional ingredients, components, or limitations described herein or otherwise useful.

Unless otherwise specified herein, all percentages and ratios of components are by weight, relative to the total weight of the final composition.

In an embodiment, the present invention relates to a hair cosmetic composition, containing, in a cosmetically acceptable carrier:
(a) from about 0.375 to about 2.55% by weight of at least one polyurethane latex polymer; and
(b) from about 1 to about 5% by weight of at least one silicone-organic polymer compound;
   all weights being relative to the weight of the composition, and wherein the weight ratio of the polyurethane latex polymer to the silicone-organic polymer compound is from about 0.15 to about 1.5.

In an embodiment, the present invention relates to a hair cosmetic composition, containing, in a cosmetically acceptable carrier:
(a) from about 0.45% to about 2.21% by weight of at least one polyurethane latex polymer; and
(b) from about 2% to about 4% by weight of at least one silicone-organic polymer compound;
   all weights being relative to the weight of the composition, and wherein the weight ratio of the polyurethane latex polymer to the silicone-organic polymer compound is from about 0.25 to about 1.1.

In another embodiment, present invention is directed to a hair cosmetic composition containing, in a cosmetically acceptable carrier:
(a) from about 0.25% to about 3.5% by weight of at least one polyurethane latex polymer; and
(b) from about 0.5% to about 6% by weight of at least one silicone-organic polymer compound;
   all weights being relative to the weight of the composition;
   wherein the at least one cosmetically acceptable carrier is selected from water, at least one organic solvent, and mixtures thereof;
   wherein the weight ratio of the polyurethane latex polymer to the silicone-organic polymer compound is less than 2.

In an embodiment, the at least one polyurethane latex polymer in the above-described compositions is selected from polyurethane-34.

In an embodiment, the at least one polyurethane latex polymer in the above-described compositions has a Young's modulus ranging from about 0.1 MPa to about 10 MPa, and a strain, under stress at 0.5 MPa, of at least about 1%.

In a certain embodiment, the at least one polyurethane latex polymer in the above-described compositions is selected from polyurethane-34, wherein polyurethane-34 has a Young's Modulus of 3 MPa and strain, under stress at 0.5 MPa, of 18.82%.

In an embodiment, the at least one silicone-organic polymer compound in the above-described compositions is dispersed in an alcoholic medium.

In an embodiment, the at least one silicone-organic polymer compound in the above-described compositions is selected from Crotonic Acid/Vinyl C8-12 Isoalkyl Esters/VA/Bis-Vinyldimethicone Crosspolymer.

In another embodiment, the at least one silicone-organic polymer compound in the above-described compositions is partially or fully neutralized form.

In an embodiment, the cosmetically acceptable carrier is selected from water, at least one organic solvent, and mixtures thereof.

In another embodiment, the cosmetically acceptable carrier comprises water, and at least one organic solvent.

In other embodiments, the at least one organic solvent that may be present in the carrier of the above-described compositions is selected from ethanol, isopropyl alcohol, butanol, propylene glycol, butylene glycol, pentylene glycol, hexylene glycol, glycerol, isododecane, mineral oil, polybutene, dipropylene glycol n-butyl ether, and mixtures thereof.

In other embodiments, the above-described compositions of the present invention further comprise a nonionic surfactant selected from fatty alcohols, alkoxylated alcohols, glyceryl esters, alkylpolyglycosides, and mixtures thereof.

In yet other embodiments, the above-described compositions of the present invention further comprise a propellant.

In certain embodiments, the above-described compositions are hair styling or hair shaping compositions.

In yet other embodiments, the above-described compositions are anti-frizz hair cosmetic compositions.

In an embodiment, the present invention relates to methods of controlling frizziness of hair comprising applying to the hair any one of the above described compositions. Such methods may further comprise the additional step of heating the hair and/or applying a smoothing action to the hair.

In some embodiments, the above-described compositions of the present invention impart frizz control benefits to hair that remain after one or more shampoo-wash cycles.

### Latex Polymers

According to various exemplary embodiments of the invention, the at least one polyurethane latex polymer is a film-forming polymer.

In various embodiments, the polyurethane latex polymer has a Young's modulus ranging from about 0.1 MPa to about 10 MPa and a strain, under stress at 0.5 MPa, of at least about 1%. In other embodiments, the polyurethane latex polymer has a Young's modulus ranging from about 10 MPa to about 6 GPa and a strain, under stress at 0.5 MPa, of less than about 5%. In at least certain embodiments, the polyurethane latex polymer may have a glass transition temperature (Tg) ranging from about −90° C. to about 40° C., and in other embodiments, the polyurethane latex polymer may have a glass transition temperature (Tg) ranging from about 40° C. to about 200° C.

In at least certain exemplary and non-limiting embodiments, the polyurethane latex polymer is a relatively soft, flexible latex polymer; in other embodiments, the polyurethane latex polymer is a relatively hard, brittle polymer, although such characteristics are not required.

As used herein, a film-forming polymer is meant to include a polymer that is capable, by itself or in the presence of an auxiliary film-forming agent, of forming a macroscopically continuous film that adheres to keratin materials, and preferably a cohesive film, better still, a film whose cohesion and mechanical properties are such that said film can be isolated and manipulated individually, for example, when said film is prepared by pouring onto a non-stick surface such as Teflon-coated or silicone-coated surface. In addition, as used herein, a non-film-forming polymer is meant to include a polymer which will not form a film at ambient temperature or below, or in other words, will only form a film at temperatures above ambient. For purposes of this disclosure, ambient temperature is taken as being below 40° C. such as in the range of 15° C. to 30° C.

In at least certain embodiments of the invention, the polyurethane latex polymers are provided in the form of aqueous dispersions prior to least one polyurethane latex polymer is provided in the form of an aqueous dispersion prior to formulating the compositions of the invention. In various embodiments, the aqueous dispersions may be obtained through an emulsion polymerization of monomers wherein the resulting latex polymers have a particle size lower than about 1 m. In at least one exemplary embodiment, a dispersion prepared by the polymerization in water of one or more monomers having a polymerizable double bond may be chosen. In another exemplary embodiment, the aqueous dispersions obtained through an emulsion polymerization may be spray-dried.

In other embodiments, the polyurethane latex polymers are produced from condensation reactions between monomers and subsequently dispersed in an aqueous medium.

Thus, the polyurethane latex polymers may, in various exemplary embodiments, exist as dispersed polymer particles in a dispersion medium, such as an aqueous dispersion medium. The latex polymers may, in certain embodiments, each be dispersed in independent dispersion media. In yet further embodiments, the latex polymers may be dispersed together in the same dispersion medium.

The dispersion medium comprises at least one solvent chosen from water. The dispersion medium may further comprise at least one solvent chosen from cosmetically acceptable organic solvents. Cosmetically acceptable organic solvents may, in various embodiments, be water-miscible, e.g. capable of forming at 25° C. a homogeneous mixture that is transparent, or substantially transparent, to the eye. For instance, cosmetically acceptable organic solvents may be chosen from lower monoalcohols, such as those containing from about 1 to 5 carbon atoms, for example ethanol and isopropanol; polyols, including glycols, such as those containing from about 2 to 8 carbon atoms, for example propylene glycol, ethylene glycol, 1,3-butylene glycol, dipropylene glycol, hexylene glycol, and glycerin; hydrocarbons, such as, for example, isododecane and mineral oil; and silicones, such as dimethicones, cyclomethicones, and cyclopentasiloxane; as well as mixtures thereof.

In at least one embodiment, the solvent of the dispersion medium consists of water. In other embodiments, the solvent of the dispersion medium consists of water and at least one cosmetically acceptable organic solvent. In further embodiments, the solvent comprises water. In yet further embodiments, the solvent of the dispersion medium primarily comprises water. For example, the solvent of the dispersion medium may, in at least certain exemplary embodiments, comprise greater than 50% water, such as greater than 55% water, greater than 60% water, greater than 65% water, greater than 70% water, greater than 75% water, greater than 80% water, greater than 85% water, greater than 90% water, greater than 95% water, greater than 96% water, greater than 97% water, greater than 98% water, or greater than 99% water.

In embodiments according to the invention, the polyurethane latex polymer particles are not soluble in the solvent of the dispersion medium, i.e. are not water soluble and/or are not soluble in the at least one cosmetically acceptable organic solvent. Accordingly, the polyurethane latex polymers retain their particulate form in the solvent or solvents chosen.

In at least certain exemplary embodiments, latex particles according to the invention may have an average diameter ranging up to about 1000 nm, such as from about 50 nm to about 800 nm, or from about 100 nm to about 500 nm. Such particle sizes may be measured with a laser granulometer (e.g. Brookhaven BI90).

In various embodiments, the polyurethane latex polymers may, independently, be neutralized, partially neutralized, or unneutralized. In exemplary embodiments where the latex polymers are neutralized or partially neutralized, the particle size may be, for example, greater than about 800 nm. In at least certain embodiments, the particulate form of the polyurethane latex polymers is retained in the dispersion medium.

In further embodiments, the polyurethane latex polymers may be chosen from uncharged and charged latex polymers. Thus, the polyurethane latex polymers may, according to various exemplary embodiments, be chosen from nonionic latex polymers, cationic latex polymers, and anionic latex polymers.

In yet further exemplary and non-limiting embodiments, the polyurethane latex polymers are provided as aqueous polyurethane dispersions comprising the reaction products of (i), (ii), and/or (iii), defined below.

Reaction product (i) may be any prepolymer according to the formula:

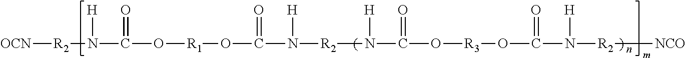

wherein R1 is chosen from bivalent radicals of a dihydroxyl functional compound, R2 is chosen from hydrocarbon radicals of an aliphatic or cycloaliphatic polyisocyanate, and R3 is chosen from radicals of a low molecular weight diol, optionally substituted with ionic groups, n ranges from about 0 to about 5, and m is greater than about 1.

Suitable dihydroxyl compounds for providing the bivalent radical R1 include those having at least two hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the invention.

The polyester diol(s) may optionally be prepared from aliphatic, cycloaliphatic, or aromatic dicarboxylic or polycarboxylic acids, or anhydrides thereof; and dihydric alcohols such as diols chosen from aliphatic, alicyclic, or aromatic diols.

The aliphatic dicarboxylic or polycarboxylic acids may be chosen from, for example: succinic, fumaric, glutaric, 2,2-dimethylglutaric, adipic, itaconic, pimelic, suberic, azelaic, sebacic, maleic, malonic, 2,2-dimethylmalonic, nonanedicarboxylic, decanedicarboxylic, dodecane [symbol] dioic, 1,3-cyclohexanedicarboxylic, 1,4-cyclo [symbol] hexane-dicarboxylic, 2,5-norboranedicarboxylic, diglycolic, thiodipropionic, 2,5-naphthalene-dicarboxylic, 2,6-naphthalene [symbol] dicarboxylic, phthalic, terephthalic, isophthalic, oxanic, o-phthalic, tetrahydrophthalic, hexahydrophthalic or trimellitic acid.

The acid anhydrides may, in further exemplary embodiments, be chosen from o-phthalic, trimellitic or succinic acid anhydride or a mixture thereof. By way of non-limiting example only, the dicarboxylic acid may be adipic acid.

The dihydric alcohols may be chosen from, for example, ethanediol, ethylene glycol, diethylene glycol, triethylene glycol, trimethylene glycol, tetraethylene glycol, 1,2-propanediol, dipropylene glycol, tripropylene glycol, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, cyclohexanedimethanol, 1,8-octanediol, 1,10-decanediol, 1,12-dodecanediol, neopentyl glycol, and mixtures thereof. The cycloaliphatic and/or aromatic dihydroxyl compounds may also be suitable as the dihydric alcohol(s) for the preparation of the polyester polyol(s).

The polyester diols may also be chosen from homopolymers or copolymers of lactones, which are, in at least certain embodiments, obtained by addition reactions of lactones or lactone mixtures, such as butyrolactone, ε-caprolactone and/or methyl-ε-caprolactone with the appropriate polyfunctional, e.g. difunctional, starter molecules such as, for example, the dihydric alcohols mentioned above. The corresponding polymers of ε-caprolactone may be chosen in at least some embodiments.

The polyester polyol, e.g. polyester diol, radical R1, may be obtained by polycondensation of dicarboxylic acids, such as adipic acid, with polyols, e.g. diols, such as hexanediol, neopentyl glycol, and mixtures thereof.

The polycarbonates containing hydroxyl groups comprise those known per se, such as the products obtained by reacting diols, such as (1,3)-propanediol, (1,4)-butanediol and/or (1,6)-hexanediol, diethylene glycol, triethylene glycol, or tetraethylene glycol with diaryl carbonates, for example diphenyl carbonate or phosgene.

Optional polyether polyols may be obtained in any known manner by reacting starting compounds which contain reactive hydrogen atoms with alkylene oxides, such as, for example, ethylene oxide; propylene oxide; butylene oxide; styrene oxide; tetrahydrofuran; or epichlorohydrin, or with mixtures of these alkylene oxides. In at least certain embodiments, the polyethers do not contain more than about 10% by weight of ethylene oxide units. For example, polyethers obtained without addition of ethylene oxide may be chosen.

Polyethers modified with vinyl polymers are also suitable according to various embodiments of the invention. Products of this type can be obtained by polymerization, for example, of styrene and acrylonitrile in the presence of polyethers, for example as described in U.S. Pat. Nos. 3,383,351; 3,304,273; 3,523,095; 3,110,695; and German patent 1 152 536.

Among the polythioethers which may be chosen include the condensation products obtained from thiodiglycol per se and/or with other glycols, dicarboxylic acids, formaldehyde, aminocarboxylic acids, and/or amino alcohols. The products obtained are either mixed polythioethers, polythioether esters, or polythio [symbol] ether ester amides, depending on the co-components.

Optional polyacetals include but are not limited to the compounds which can be prepared from aldehydes, for example formaldehyde, and from glycols, such as diethylene glycol, triethylene glycol, ethoxylated 4,4′-(dihydroxy)diphenyl-dimethylmethane, and (1,6)-hexane [symbol] diol. Polyacetals useful according to various non-limiting embodiments of the invention can also be prepared by polymerization of cyclic acetals.

Optional polyhydroxy polyesteramides and polyamines include, for example, the mainly linear condensation products obtained from saturated or unsaturated, polybasic carboxylic acids or anhydrides thereof, and from saturated or unsaturated, polyvalent amino alcohols, from diamines, or from polyamines, as well as mixtures thereof.

Optional monomers for the production of polyacrylates having hydroxyl functionality comprise acrylic acid, methacrylic acid, crotonic acid, maleic anhydride, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, glycidyl acrylate, glycidyl methacrylate, 2-isocyanatoethyl acrylate, and 2-isocyanatoethyl methacrylate.

Mixtures of dihydroxy compounds can also be chosen.

Optional polyisocyanates for providing the hydrocarbon-based radical R2 include, for example, organic diisocyanates having a molecular weight ranging from about 100 to about 1500, such as about 112 to about 1000, or about 140 to about 400.

Optional diisocyanates are those chosen from the general formula R2(NCO)2, in which R2 represents a divalent aliphatic hydrocarbon group comprising from about 4 to 18 carbon atoms, a divalent cycloaliphatic hydrocarbon group comprising from about 5 to 15 carbon atoms, a divalent araliphatic hydrocarbon group comprising from about 7 to 15 carbon atoms, or a divalent aromatic hydrocarbon group comprising from about 6 to 15 carbon atoms. Examples of the organic diisocyanates which may be chosen include, but are not limited to, tetramethylene diisocyanate, 1,6-hexamethylene diisocyanate, dodecamethylene diisocyanate, cyclohexane-1,3-diisocyanate and cyclohexane-1,4-diisocyanate, 1-isocyanato-3-isocyanato [symbol] methyl-3,5,5-trimethylcyclohexane (isophorone diisocyanate or IPDI), bis(4-isocyanatocyclohexyl)-methane, 1,3-bis(isocyanatomethyl)cyclohexane and 1,4-bis(isocyanatomethyl)cyclohexane and bis(4-isocyanato-3-methylcyclohexyl)methane. Mixtures of diisocyanates can also be used.

In at least certain embodiments, diisocyanates are chosen from aliphatic and cycloaliphatic diisocyanates. For example, 1,6-hexamethylene diisocyanate, isophorone diisocyanate, and dicyclohexylmethane diisocyanate, as well as mixtures thereof may be chosen.

The use of diols, for example low molecular weight diols, R3, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In certain exemplary embodiments, the compounds contain only aliphatic groups. The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxy [symbol] cyclo [symbol] hexyl)-propane), and mixtures thereof. For example, R3 may be derived from neopentyl glycol.

Optionally, the low molecular weight diols may contain ionic or potentially ionic groups. Suitable low molecular weight diols containing ionic or potentially ionic groups may be chosen from those disclosed in U.S. Pat. No. 3,412,054. In various embodiments, compounds may be chosen from dimethylohbutanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In at least certain exemplary and non-limiting embodiments, the low molecular weight diols containing ionic or potentially ionic groups are not used.

Reaction product (ii) may be chosen from at least one chain extender according to the formula:

H2N—R4-NH2

wherein R4 is chosen from alkylene or alkylene oxide radicals, said radicals not being substituted with ionic or potentially ionic groups.

Reaction product (ii) may optionally be chosen from alkylene diamines, such as hydrazine, ethylene [symbol] diamine, propylenediamine, 1,4-butylenediamine and piperazine; and alkylene oxide diamines such as dipropylamine diethylene glycol (DPA-DEG available from Tomah Products, Milton, Wis.), 2-methyl-1,5-pentanediamine (Dytec A from DuPont), hexanediamine, isophorone [symbol] diamine, and 4,4-methylenedi(cyclohexylamine), and the DPA-series of ether amines available from Tomah Products, Milton, Wis., including dipropylamine propylene glycol, dipropylamine dipropylene glycol, dipropylamine tripropylene glycol, dipropylamine poly(propylene glycol), dipropylamine ethylene glycol, dipropylamine poly(ethylene glycol), dipropylamine 1,3-propanediol, dipropylamine 2-methyl-1,3-propanediol, dipropylamine 1,4-butanediol, dipropylamine 1,3-butanediol, dipropylamine 1,6-hexanediol and dipropylamine cyclohexane-1,4-dimethanol, and mixtures thereof.

Reaction product (iii) may be chosen from at least one chain extender according to the formula:

H2N—R5-NH2

wherein R5 is chosen from alkylene radicals substituted with ionic or potentially ionic groups. In at least certain exemplary embodiments, the compounds may have an ionic or potentially ionic group and two isocyanate-reactive groups.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Specific compounds include diaminosulphonates, such as for example the sodium salt of N-(2-aminoethyl)-2-aminoethanesulphonic acid (AAS) or the sodium salt of N-(2-aminoethyl)-2-aminopropionic acid.

In at least certain embodiments, R5 represents an alkylene radical substituted with sulphonic acid or sulphonate groups. By way of example only, the compound is chosen from sodium salts of N-(2-aminoethyl)-2-aminoethanesulphonic acid.

By way of non-limiting example, such latexes include, but are not limited to, aqueous polyurethane dispersions comprising a reaction product of a prepolymer such as, for example, those sold under the BAYCUSAN® name by Bayer such as, for example, BAYCUSAN® C1000 (INCI name: Polyurethane-34), BAYCUSAN® C1001 (INCI name: Polyurethane-34), BAYCUSAN® C1003 (INCI name: Polyurethane-32), BAYCUSAN® C1004 (INCI name: Polyurethane-35) and BAYCUSAN® C1008 (INCI name: Polyurethane-48). In various exemplary embodiments, polyurethane latexes may be chosen from, but are not limited to, aqueous polyurethane dispersion of Isophthalic Acid/Adipic Acid/Hexylene Glycol/Neopentyl glycol/Dimethylolpropanoic Acid/Isophorone Diisocyanate copolymer (INCI name: Polyurethane-1, such as LUVISET® P.U.R, BASF), polycarbonate polyurethane, aliphatic polyurethane and aliphatic polyester polyurethane (such as the NEOREZ® series, DSM, such as NEOREZ® R989, INCI name: Polycarbamyl Polyglycon Ester).

In certain embodiments, the polyurethane latex polymer of the present invention is preferably chosen from Polyurethane-34, commercially available as aqueous dispersions under the tradenames BAYCUSAN® C1000 or BAYCUSAN® C1001 from the supplier Bayer Material Science.

The dispersions containing the polyurethane latex polymers of the present invention may be employed in the composition of the present invention, in an amount, of from about 1 to about 10% by weight, preferably from about 1.25 to about 7.5% by weight, more preferably from about 1.5 to about 6.5% by weight, including all ranges and subranges there-between, relative to the weight of the composition.

The at least one polyurethane latex polymer of the present invention is present in the composition of the present invention, in an amount, of from about 0.25 to about 3.5% by weight, preferably from about 0.375 to about 2.55% by weight, more preferably from about 0.45 to about 2.21% by weight, including all ranges and subranges there-between, relative to the weight of the composition. These weights are on a dry weight basis.

In some embodiments, the hair cosmetic composition of the present invention further comprises acrylate latex polymers.

In certain embodiments, the hair cosmetic composition of the present invention is substantially free of acrylate latex polymers.

In yet other embodiments, the hair cosmetic composition of the present invention does not contain acrylate latex polymers.

The term “substantially free of acrylate latex polymers” as it is used herein means that while it is preferred that no acrylate latex polymers be present in the composition, it is possible to have very small amounts of acrylate latex polymers in the compositions of the invention provided that these amounts do not materially affect at least one, preferably most, of the advantageous properties of the compositions of the invention. In particular, “substantially free of acrylate latex polymers” means that acrylate latex polymers can be present in the composition, on a dry weight basis, in an amount of less than about 1.5% by weight, or less than about 1% by weight, or less than about 0.5% by weight, or less than about 0.05% by weight, including all ranges and subranges there-between, relative to the weight of the composition.

Acrylate latex polymers are those resulting from the homopolymerization or copolymerization of monomers chosen from (meth)acrylics, (meth)acrylates, (meth)acrylamides and/or vinyl homopolymers or copolymers. The term “(meth)acryl” and variations thereof, as used herein, means acryl or methacryl.

The (meth)acrylic monomers may be chosen from, for example, acrylic acid, methacrylic acid, citraconic acid, itaconic acid, maleic acid, fumaric acid, crotonic acid, and maleic anhydride. Additional non-limiting examples of (meth)acrylic monomers include C1-C8 alkyl (meth)acrylic, such as, for example, methyl (meth)acrylic, ethyl (meth)acrylic, propyl (meth)acrylic, isopropyl (meth)acrylic, butyl (meth)acrylic, tert-butyl (meth)acrylic, pentyl(meth) acrylic, isopentyl (meth)acrylic, neopentyl (meth)acrylic, hexyl (meth)acrylic, isohexyl (meth)acrylic, 2-ethylhexyl (meth)acrylic, cyclohexyl (meth)acrylic, isohexyl (meth)acrylic, heptyl (meth)acrylic, isoheptyl (meth)acrylic, octyl (meth)acrylic, isooctyl (meth)acrylic, as well as combinations of any of the above.

The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. Additional and non-limiting examples include C1-C8 alkoxy (meth)acrylates, such as methoxy (meth)acrylate, ethoxy (meth)acrylate, propyl oxide (meth)acrylate, isopropyl oxide (meth)acrylate, butyl oxide (meth)acrylate, tert-butyl oxide (meth)acrylate, pentyl oxide (meth) acrylate, isopentyl oxide (meth)acrylate, neopentyl oxide (meth)acrylate. The esters may be, by way of non-limiting example, C2-C6 hydroxy alkyl (meth)acrylates, such as hydroxy ethyl (meth)acrylate, 2-hydroxypropyl (meth)acrylate, glycidyl (meth)acrylate, ethylene glycol di(meth)acrylate, polyethylene glycol mono(meth)acrylate, 1,4-butane diol di(meth)acrylate, 1,6,hexane diol di(meth)acrylate, and any combination thereof. The esters may be, by way of non-limiting example, aryl (meth)acrylates such as benzyl (meth)acrylate, phenyl (meth)acrylate, and any combination thereof. The esters can further contain amino groups such as aminoethyl (meth)acrylate, N,N-dimethylaminoethyl (meth)acrylate, N,N-dimethylaminopropyl (meth)acrylate, N,N-dimethylaminodimethylpropyl (meth)acrylate, N,N-diethyleaminoethyl (meth)acrylate, and N,N,N-trimethylaminoethyl (meth)acrylate; and salts of the ethylenic amines.

According to at least certain exemplary embodiments, the alkyl group of the esters may be either fluorinated or perfluorinated, e.g. some or all of the hydrogen atoms of the alkyl group are substituted with fluorine atoms. The monomers can also be fluorine-containing monomers, such as, by way of non-limiting example, trifluoroethyl methacrylate, 2,2,3,3-tetrafluoropropyl methacrylate, 2,2,3,3,4,4-hexafluorobutyl methacrylate, perfluorooctyl methacrylate and perfluorooctyl acrylate; and silicone macromonomers.

The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, α-methylstyrene, chlorostyrene, alkylstyrene, divinylbenzene and diallyl phthalate, and combination thereof. Other non-limiting ionic monomers can include para-styrensulfonic, vinylsulfonic, 2-(meth)acryloyloxyethylsulfonic, 2-(meth)acrylamido-2-methylpropylsulfonic acids.

The list of monomers given is not limiting, and it should be understood that it is possible to use any monomer known to those skilled in the art which includes acrylic and/or vinyl monomers (including monomers modified with a silicone chain).

Silicone acrylic polymers may also optionally be used as vinyl polymer in at least one exemplary and non-limiting embodiment.

In at least certain, non-limiting exemplary embodiments, acrylic latex polymers may be chosen from aqueous dispersions of Methacrylic Acid/Ethyl Acrylate copolymer (INCI: Acrylates Copolymer, such as LUVIFLEX® Soft by BASF), PEG/PPG-23/6 Dimethicone Citraconate/C10-30 Alkyl PEG-25 Methacrylate/Acrylic Acid/Methacrylic Acid/Ethyl Acrylate/Tri methylolpropane PEG-15 Triacrylate copolymer (INCI: Polyacrylate-2 Crosspolymer, such as FIXATE SUPERHOLD™ by Lubrizol), Styrene/Acrylic copolymer (such as NEOCRYL® A-1120, DSM), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as DAITOSOL 5000AD, Daito Kasei Kogyo), Vinyl Acetate Acrylic Ester Copolymer (INCI name: Acrylates/VA Copolymer, such as VINYSOL 2140, Daido Chemical) and Acrylates Copolymers, such as those known under the tradename ACULYN™ 33 (Dow Chemical), under the tradename LUVIMER® MAE (BASF), or under the tradename BALANCE CR (AKZO NOBEL), Styrene/Acrylates Copolymer, known under the tradename JONCRYL 77 from BASF, Styrene/Acrylates/Ammonium Methacrylate Copolymer, known under the tradename SYNTRAN PC5620 CG from Interpolymer, and mixtures thereof.

### Silicone-Organic Polymer Compound

The at least one silicone-organic polymer compound of the present invention includes, but is not limited to, a silicone polyvinyl acetate compound.

The silicone-organic polymer hybrid compound of the present invention may also be chosen from a cross-linked anionic copolymer comprised of organic polymer blocks and silicone blocks, resulting in a multiblock polymer structure.

In particular, the silicone-organic polymer compound of the present invention may be chosen from cross-linked anionic copolymers comprising at least one cross-linked polysiloxane structural unit. Examples of these polymers have been described in the PCT publication, WO2011069786, published Jun. 16, 2011.

A particularly preferred silicone-organic polymer compound of the present invention is a compound having the INCI name of Crotonic Acid/Vinyl C8-12 Isoalkyl Esters/VA/Bis-Vinyldimethicone Crosspolymer which is a copolymer of Crotonic Acid, vinyl C8-12 isoalkyl esters and Vinyl Acetate crosslinked with bis-vinyldimethicone. This compound is commercially available from the company Wacker Chemie AG as an alcoholic dispersion under the tradename Wacker Belsil® P1101 (may also be known under the tradename Wacker Belsil® P101). Crotonic Acid/Vinyl C8-12 Isoalkyl Esters/VA/Bis-Vinyldimethicone Crosspolymer is also known by the technical name of Crotonic Acid/Vinyl C8-12 Isoalkyl Esters/VA/divinyldimethicone Crosspolymer.

An alcoholic solution containing the at least one silicone-organic polymer compound may be employed in the composition of the present invention in an amount of from about 1 to about 12% by weight, such as from about 2 to about 10% by weight, or such as from about 4 to about 8% by weight, including all ranges and subranges there-between, relative to the weight of the composition.

The at least one silicone-organic polymer compound is present in the composition of the present invention in an amount of from about 0.5 to about 6% by weight, preferably from about 1 to about 5% by weight, or more preferably from about 2 to about 4% by weight, including all ranges and subranges there-between, relative to the weight of the composition. These weights are on a dry weight basis.

The silicone-organic polymer compound is neutralized (fully or partially) with a neutralizing agent in order to remain solubilized in the composition of the present invention. Representative examples of neutralizing agent useful for this purpose include AMP (aminomethyl propanol), AMPD (aminomethyl propanediol), TIPA (triisopropanol amine), sodium hydroxide, potassium hydroxide, Dimethylstearylamine, Dimethyl/tallowamine lysine, ornithine, arginine, monoethanolamine, triethanolamine, calcium hydroxide, calcium bicarbonate, and mixtures thereof. The amount of neutralizer is selected on criteria that include the desired pH of the composition. Thus, the amount of neutralizer generally ranges from greater than 0 (e.g., about 0.01%) to about 3%, in some embodiments from 0.05% to about 2%, in other embodiments from 0.05% to about 0.5% by weight, including all ranges and subranges there-between, relative to the weight of the composition.

In certain embodiments of the present invention, the silicone-organic polymer compound is partially neutralized, that is from 80% to less than 100% neutralized. For example, the silicone-organic polymer compound can be 80%, or 85%, or 90%, or 95% neutralized. Thus, in certain embodiments, the amount of the neutralizing agent is such that a desired of degree of neutralization can be achieved.

In other embodiments of the present invention, the silicone-organic polymer compound is 100% neutralized.

In yet other embodiments of the present invention, the silicone-organic polymer compound is at least 80% neutralized.

### Ratio of Polyurethane Latex Polymer to Silicone-Organic Polymer Compound

In some embodiments of the present invention, the weight ratio, on a dry weight basis, of the polyurethane latex polymer to the silicone-organic polymer compound is less than about 2, or ranges from about 0.1 to about 1.75 or preferably from about 0.15 to about 1.5 or more preferably from about 0.25 to about 1.1, including all ranges and subranges there-between.

In other embodiments, the weight ratio, on a dry weight basis, of the polyurethane latex polymer to the silicone-organic polymer compound ranges from about 0.235 to about 1.06, or is at about 0.25, or about 0.4, or about 0.5, or about 0.8, or about 1.

In preferred embodiments, the weight ratio, on a dry weight basis, of the polyurethane latex polymer to the silicone-organic polymer compound is at about 2:4 (or 0.5) or at about 2:2 (or 1) or at about 1:2.5 (or 0.4).

### Organic Solvents

The compositions of the present invention comprise at least one organic solvent.

Suitable organic solvents may be chosen from volatile and nonvolatile organic solvents.

Suitable organic solvents are typically C1-C4 lower alcohols, glycols, polyols, polyol ethers, hydrocarbons, and oils. Examples of organic solvents include, but are not limited to, ethanol, isopropyl alcohol, benzyl alcohol, phenyl ethyl alcohol, propylene glycol, pentylene glycol, hexylene glycol, glycerol, and mixtures thereof.

Other suitable organic solvents include glycol ethers, for example, ethylene glycol and its ethers such as ethylene glycol monomethyl ether, ethylene glycol monopropyl ether, ethylene glycol monobutyl ether, propylene glycol and its ethers, such as propylene glycol monomethyl ether, propylene glycol monopropyl ether, propylene glycol monobutyl ether, dipropylene glycol and diethylene glycol alkyl ethers, such as diethylene glycol monoethyl ether, diethylene glycolmonobutyl ether, and dipropylene glycol n-butyl ether. Glycol ethers are commercially available from The Dow Chemical Company under the DOW E-series and DOW P-series. One preferred glycol ether for use in the present invention is dipropylene glycol n-butyl ether, known under the tradename of DOWANOL DPnB.

Suitable organic solvents also include synthetic oils and hydrocarbon oils include mineral oil, petrolatum, and C₁₀-C₄₀ hydrocarbons which may be aliphatic (with a straight, branched or cyclic chain), aromatic, arylaliphatic such as paraffins, iso-paraffins, isododecanes, aromatic hydrocarbons, polybutene, hydrogenated polyisobutene, hydrogenated polydecene, polydecene, squalene, petrolatum and isoparaffins, silicone oils, fluoro oils and mixtures, thereof.

The term “hydrocarbon-based oil” or “hydrocarbon oil” refers to oil mainly containing hydrogen and carbon atoms and possibly oxygen, nitrogen, sulfur and/or phosphorus atoms. Representative examples of hydrocarbon-based oils include oils containing from 8 to 16 carbon atoms, and especially branched C8-C16 alkanes (also known as isoparaffins), for instance isododecane (also known as 2,2,4,4,6-pentamethylheptane), isodecane and isohexadecane.

Examples of silicone oils that may be useful in the present invention include nonvolatile silicone oils such as polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and dimethicones or phenyltrimethicones with a viscosity of less than or equal to 100 cSt.

Other representative examples of silicone oils that may be useful in the present invention include volatile silicone oils such as linear or cyclic silicone oils, and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of fluoro oils that may be suitable for use in the present invention include volatile fluro oils such as nonafluoromethoxybutane and perfluoro-methylcyclopentane.

The amount of the organic solvent/compound present in the compositions of the present invention can range from about 0.5% to about 95%, or from about 0.5% to about 80%, or from about 0.5% to about 60%, or from about 0.5% to about 40%, or from about 0.5% to about 30%, or from about 0.5% to about 20%, and in some embodiments, from about 0.5% to about 15%, by weight, or preferably from about 1% to about 10%, by weight, or more preferably from about 1.5% to about 8%, by weight, or from about 2% to about 6%, by weight, including all ranges and subranges there-between, relative to the total weight of the composition.

In some embodiments, the amount of the organic solvent/compound present in the compositions of the present invention is at about 2%, about 2.5%, about 3%, about 3.5%, about 4%, about 4.5%, about 5%, about 5.5% or about 6% by weight, including all ranges and subranges there-between, relative to the total weight of the composition.

In certain embodiments, compositions of the present invention comprise both water and organic solvents/compounds selected from volatile organic solvents, non-volatile organic solvents, and mixtures thereof.

Preferred examples of organic solvents/compounds include volatile organic solvents such as C2 to C4 mono-alcohols, such as ethanol, isopropyl alcohol, butanol, polyols such as C2-C6 glycols e.g., propylene glycol, butylene glycol, pentylene glycol, hexylene glycol, glycerol, isododecane, volatile polyol ethers, volatile glycol ethers, acetone, propylene carbonate, benzyl alcohol, and mixtures thereof. In certain embodiments, it is preferred that the amount of volatile organic solvent/compound does not exceed 55% by weight, relative to the weight of the composition of the present invention.

In other certain embodiments, it is preferred that the amount of volatile organic solvent/compound does not exceed 20% by weight, relative to the weight of the composition of the present invention.

In yet other certain embodiments, it is preferred that the amount of volatile organic solvent/compound does not exceed 10% by weight, relative to the weight of the composition of the present invention.

In preferred embodiments, the amount of volatile organic solvent/compound does not exceed 6% by weight, relative to the weight of the composition of the present invention.

Other preferred examples of organic solvents/compounds include nonvolatile organic solvents such as hydrocarbons such as straight chain hydrocarbons, nonvolatile silicone oils, mineral oil, polybutene, hydrogenated polyisobutene, hydrogenated polydecene, polydecene, squalene, petrolatum, isoparaffins, nonvolatile glycol ethers, and mixtures, thereof.

In certain embodiments, it is preferred that the amount of nonvolatile organic solvent/compound does not exceed 40% by weight, relative to the weight of the composition of the present invention.

In other certain embodiments, it is preferred that the amount of nonvolatile organic solvent/compound does not exceed 20% by weight, relative to the weight of the composition of the present invention.

In yet other certain embodiments, it is preferred that the amount of nonvolatile organic solvent/compound does not exceed 10% by weight, relative to the weight of the composition of the present invention.

In preferred embodiments of the present invention, the at least one organic solvent is chosen from ethanol, glycol ether, for example, dipropylene glycol n-butyl ether, known under the tradename of DOWANOL DPnB, isododecane, mineral oil, propylene glycol, pentylene glycol, hexylene glycol, glycerol, and mixtures thereof.

In certain embodiments of the present invention, the at least one organic solvent is chosen from ethanol.

The organic solvents may also comprise the solvent of the dispersion medium employed to disperse the latex polymers and silicone organic polymer compound of the present invention.

In some embodiments, the cosmetically acceptable carrier in the compositions of the present invention contains water in the amount of about 95%, 90%, 80%, 70%, 60%, 50%, 40%, 30%, 20%, 10%, 5% by weight or less, based on the total weight of the compositions. Additionally, the cosmetically acceptable carrier in the compositions of the present invention can contain water in the amount of from about 20% to about 95% by weight, or from about 50% to about 90% by weight, or from about 60% to about 80% by weight, relative to the weight of the compositions.

In other embodiments, the cosmetically acceptable carrier in the compositions of the present invention contains water in the amount of at least about 95%, 90%, 80%, 70%, 60%, 50%, 40%, 30%, 20%, 10%, 5% by weight or less, based on the total weight of the compositions.

In yet some other embodiments, the cosmetically acceptable carrier of the present invention does not comprise water that is added as a separate ingredient, by itself, into the compositions of the present invention, such that water is present in the compositions of the present invention when it accompanies one or more ingredients of a raw material that is added into the compositions of the invention.

The compositions described above are useful for application onto keratinous substrates such as hair on the head of human individuals.

Thus, the compositions of the present invention can be made into various cosmetic products such hair care products, hair styling products and make up products.

Representative types of hair care compositions, including hair cosmetic and styling compositions, of the present invention include compositions for shaping the hair, maintaining the shape of the hair, styling products (e.g., gels, creams, milks, pastes, waxes, ointments, serums, foams, hair lotions, mousses, pump-sprays, non-aerosol sprays and aerosol sprays), pre-treatments and post-treatments for color protection, conditioning or protection from heat damage, leave-in hair treatments, rinse-off hair treatments, combination shampoo/styling compositions and hair volumizing compositions.

The compositions of the present invention can be in the form of an aqueous composition or an emulsion, such as a lotion or cream.

In one embodiment, the composition of the present invention is in the form of a non-aerosol spray, preferably containing a volatile organic solvent/compound.

In one embodiment, the composition of the present invention is in the form of a cream.

### Auxiliary Ingredients

The compositions of the present invention may further comprise auxiliary ingredients selected from propellants, rheology modifiers, surfactants, lipohilic compounds, skin and hair active agents, sunscreens, preservatives, fragrances, pH adjusting agents, and mixtures thereof.

In one embodiment, the composition of the present invention can be in the form of an aerosol spray, comprising a propellant.

Representative examples of propellants include C 3 to C 5 alkanes such as n-butane, isobutane, and propane, dimethyl ether (available commercially from Harp Intl under the tradename HARP DME), C2-C5 halogenated hydrocarbons, e.g., 1,1-difluoroethane (available commercially from DuPont under the tradename DYMEL 152a), difluoroethane, chlorodifluoroethane, chlorodifluoromethane, air, nitrogen, carbon dioxide, and mixtures thereof. The amount of the propellant can range from about 3 to about 90%, and in some embodiments from about 3 to about 60%, by weight, or such as from about 3 to about 20% by weight, or such as from about 3 to about 10% by weight, or such as from about 3 to about 6% by weight, including all ranges and subranges there-between, relative to the weight of the composition.

Accordingly, the compositions of the present invention may contain at least one auxiliary ingredient, which as those skilled in the cosmetics art will appreciate, is chosen based on several criteria, including for example, the type of product and its intended use and effect, compatibility with the other ingredients, and aesthetic appeal. Representative types of such additional ingredients include rheology modifiers (also known as gelling agents or thickeners), nonionic surfactants, lipophilic compounds such as oils and waxes, and hair and skin active ingredients. Examples of these ingredients are described herein.

### Rheology Modifiers

Broadly, the rheology modifier(s) that may be useful in the practice of the present invention include those conventionally used in cosmetics such as polymers of natural origin and synthetic polymers.

Representative rheology-modifying agents that may be used in the practice of the present invention include nonionic, anionic, cationic, and amphoteric polymers, and other rheology modifiers such as cellulose-based thickeners (e.g., hydroxyethylcellulose, hydroxypropylcellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum, scleroglucan gum, etc.), and gums derived from plant exudates (e.g., gum arabic, ghatti gum, karaya gum, gum tragacanth, carrageenan gum, agar gum and carob gum), pectins, alginates, and starches, crosslinked homopolymers of acrylic acid or of acrylamidopropane-sulfonic acid, associative polymers, non-associative thickening polymers, and water-soluble thickening polymers.

In some embodiments, the rheology-modifying agent includes a polymer chosen from nonionic, anionic, cationic and amphoteric amphiphilic polymers.

The rheology-modifying agents may also be chosen from associative celluloses include quaternized cationic celluloses and quaternized cationic hydroxyethylcelluloses modified by groups containing at least one hydrophobic chain, such as alkyl, arylalkyl or alkylaryl groups containing at least 8 carbon atoms, and mixtures thereof.

The alkyl radicals carried by the above quaternized celluloses or hydroxyethylcelluloses may, in various embodiments, comprise from 8 to 30 carbon atoms. The aryl radicals may, for example, denote the phenyl, benzyl, naphthyl or anthryl groups. Representative examples of quaternized alkylhydroxy-ethylcelluloses containing a C8-C30 hydrophobic chain include the products Quatrisoft LM 200®, Quatrisoft LM-X 529-18-A®, Quatrisoft LM-X 529-18B® (C12 alkyl) and Quatrisoft LM-X 529-8® (Ci8 alkyl) sold by Amerchol and the products Crodacel QM®, Crodacel QL® (C12 alkyl) and Crodacel QS® (Ci8 alkyl) sold by Croda.

Representative examples of nonionic cellulose derivatives include hydroxyethylcelluloses modified by groups comprising at least one hydrophobic chain, such as alkyl, arylalkyl or alkylaryl groups, or their blends, and in which the alkyl groups are, for example, C8-C22 alkyl groups, such as the product Natrosol Plus Grade 330 CS® (C16 alkyls) sold by Aqualon or the product Bermocoll EHM 100® sold by Berol Nobel.

Representative examples of cellulose derivatives modified by alkylphenyl polyalkylene glycol ether groups include the product Amercell Polymer HM-1500® sold by Amerchol.

The rheology-modifying agent is typically present in an amount ranging from about 0.01% to about 10% by weight, in some embodiments from about 0.1% to about 5% by weight, including all ranges and subranges there-between, relative to the weight of the composition.

The compositions of the present invention may further comprise compounds such as gellifying and viscosity modifying agents which may aid in improving the viscosity of the compositions.

### Surfactants

The compositions of the present invention can further comprise at least one surfactant selected from nonionic surfactants.

Non-limiting examples of nonionic surfactants includes alkoxylated derivatives of the following: fatty alcohols, alkyl phenols, fatty acids, fatty acid esters and fatty acid amides, wherein the alkyl chain is in the C12-50 range, typically in the C16-40 range, more typically in the C24 to C40 range, and having from about 1 to about 110 alkoxy groups. The alkoxy groups are selected from the group consisting of C2-C6 oxides and their mixtures, with ethylene oxide, propylene oxide, and their mixtures being the typical alkoxides. The alkyl chain may be linear, branched, saturated, or unsaturated. Of these alkoxylated non-ionic surfactants, the alkoxylated alcohols are typical, and the ethoxylated alcohols and propoxylated alcohols are more typical. The alkoxylated alcohols may be used alone or in mixtures with those alkoxylated materials disclosed herein-above.

Examples of fatty alcohols are lauryl alcohol, cetyl alcohol, stearyl alcohol, cetearyl alcohol, and behenyl alcohol.

Commercially available nonionic surfactants are Brij® nonionic surfactants from Croda, Inc., Edison, N.J. Typically, Brij® is the condensation products of aliphatic or fatty alcohols with from about 1 to about 54 moles of ethylene oxide, the alkyl chain of the alcohol being typically a linear chain and having from about 8 to about 22 carbon atoms, for example, Brij® 72 (i.e., Steareth-2) and Brij® 76 (i.e., Steareth-10).

Also useful herein as nonionic surfactants are alkyl glycosides or alkylpolyglycosides (alkylpolyglucosides) which are the condensation products of long chain alcohols, which are the condensation products of long chain alcohols, e.g. C8-C30 alcohols, with sugar or starch polymers. These compounds can be represented by the formula (S)n-O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C8-C20 alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein sucrose ester surfactants such as sucrose cocoate and sucrose laurate.

Other nonionic surfactants suitable for use in the present invention are glyceryl esters and polyglyceryl esters, including but not limited to, glyceryl monoesters, typically glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C16-C22 saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C16-C22 saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Croda, Inc., Edison, N.J.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use as nonionic surfactants are alkoxylated derivatives of glyceryl esters, sorbitan esters, and alkyl polyglycosides, wherein the alkoxy groups are selected from the group consisting of C2-C6 oxides and their mixtures, with ethoxylated or propoxylated derivatives of these materials being typical. Nonlimiting examples of commercially available ethoxylated materials include TWEEN® (ethoxylated sorbitan mono-, di- and/or tri-esters of C12 to C18 fatty acids with an average degree of ethoxylation of from about 2 to 20).

One type of preferred nonionic surfactants include alkoxylated alcohols such as a polyethylene derivative of hydrogenated castor oil, for example, PEG-40 hydrogenated castor oil, commercially available from the company Cognis (BASF) under the tradename Eumulgin® HRE 40 or Cremophor® CO 40.

The at least one nonionic surfactant is typically present in an amount from about 0.5 by weight to about 30% by weight, typically in an amount from about 1 by weight to about 20% by weight, more typically from about 0.5 by weight to 15% by weight, and even more typically from about 0.5 by weight to 10% by weight, or from about 0.5 to about 5% by weight, or from about 0.5 to about 2% by weight, including all ranges and subranges there-between, relative to the weight of the composition of the present invention.

### Lipophilic Compounds

The compositions of the present invention can further comprise at least one lipophilic compound which can be chosen from plant oils, fatty esters, waxes, fatty acids and salts thereof, lipophilic vitamins and esters thereof, organic sunscreens, phospholipids, and mixtures thereof.

Non-limiting examples of plant oils include olive oil, avocado oil, coconut oil, safflower oil, almond oil, castor oil, jojoba oil, peanut oil, sesame oil, hazelnut oil, sunflower oil, apricot kernel oil, grapeseed oil, palm oil, argan oil, squalane and pracaxi oil.

Non-limiting examples of waxes include paraffin wax, beeswax, candelilla wax, carnauba wax, jasmine wax, jojoba wax and mimosa wax.

Suitable fatty acids include those containing from 8 to 30, preferably from 12 to 24 carbon atoms, and carboxylate salts of fatty acids. The sodium, potassium, ammonium, calcium and magnesium carboxylates of fatty acids listed are typical examples of the carboxylate salts of the fatty acids.

Non-limiting preferred fatty esters include esters formed from fatty acids and C₁₋₁₀ alcohols and esters formed from the fatty alcohols as defined hereabove and C₁₋₁₀ carboxylic acids.

In addition, non-limiting specific examples of lipophilic compounds include isopropyl palmitate, capric/caprylic triglyceride, isodecyl neopentanoate, polylsobutylene, Phloretin, Ellagic acid, Vitamin D, Vitamin E, Vitamin E Acetate, Vitamin A, Vitamin A Palmitate, 2-oleamido-1,3-octadecanediol, octyl methoxycinnamate, octyl salicylate, 18-Methyleicosanoic acid, and mixtures thereof. Other types of lipophiles include organic sunscreens, phospholipids, and other water-insoluble vitamins.

According to one embodiment, the at least one lipophilic compound is chosen from plant oils, waxes, fatty acids having at least 12 carbon atoms, fatty esters and mixtures thereof.

According to another embodiment, the at least one lipophilic compound comprises fragrance oils.

The at least one lipophilic compound is present in the composition of the present invention in an amount of from about 0.1 to about 20% by weight, such as from about 0.3 to about 10% by weight, and from about 0.5 to about 5% by weight, including all ranges and subranges there-between, relative to the total weight of the composition.

As skin and hair active agents that may be used in the composition of the present invention, examples that may be mentioned include moisturizers, for example, protein hydrolysates and sugar derivatives; natural and plant extracts; anti-inflammatory agents; antioxidants; anti-wrinkle agents; procyannidol oligomers; vitamins, for instancevitamin A (retinol), vitamin C (ascorbic acid), vitamin E (tocopherol), vitamin B5 (panthenol), vitamin B3 (niacinamide), derivatives of these vitamins (especially esters) and mixtures thereof; urea; caffeine; depigmenting agents such as kojic acid, hydroquinone and caffeic acid; salicylic acid and its derivatives; α-hydroxy acids such as lactic acid and glycolic acid and derivatives thereof; β-hydroxy acids, α-keto acids, β-keto acids, retinoids such as carotenoids and vitamin A derivatives; sunscreens; self-tanning agents; hydrocortisone; melatonin; algal, fungal, plant, yeast or bacterial extracts; enzymes; DHEA and its derivatives and metabolites; antibacterial active agents, for instance 2,4,4′-trichloro-2′-hydroxydi-phenyl ether (or Triclosan), 3, 4,4′-trichloro-carbanilide (or Triclocarban); mattifying agents and mixtures thereof.

Non-limiting examples of sunscreens include benzophenones, bornelone, butyl PABA, cinnamidopropyl trimethyl ammonium chloride, disodium distryrylbiphenyl disulfonate, PABA, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, ethylhexyl dimethyl PABA, red petrolatum, and mixtures thereof.

Non-limiting examples of preservatives include polyvinyl alcohol, phenoxyethanol, benzyl alcohol, methyl paraben, propyl paraben and mixtures thereof.

Non-limiting examples of pH adjusting agents include potassium acetate, sodium carbonate, sodium hydroxide, phosphoric acid, succinic acid, sodium citrate, citric acid, boric acid, lactic acid, sodium hydrogen carbonate and mixtures thereof.

Yet other examples of auxiliary ingredients that may be present in the inventive compositions include fragrances, preservatives, colorants, glitter, fillers/powders, buffers, chelators (such as EDTA and salts thereof, particularly sodium and potassium salts), reducing agents, plasticizers, softeners, antifoaming agents, inorganic colloids, peptizing agents, pearlescent agents, penetrants, opacifying agents, and any other additive or adjuvant conventionally used in cosmetic compositions intended to be applied to the hair. The compositions may further contain polymers other than the silicone-organic polymer hybrid compound of the invention, provided that they are compatible with the other ingredients therein.

The at least one auxiliary ingredient is present in the composition in a preferred amount of from about 0.001 to about 50% and more preferably from about 0.01 to about 20% by weight, including all ranges and subranges there-between, relative to the weight of the composition.

One embodiment of the present invention is a hair cosmetic composition comprising from about 0.25% to about 3.5% by weight, including all ranges and subranges there-between, relative to the weight of the composition, of at least one polyurethane latex film-forming polymer selected from polyurethane-34, from about 0.5% to about 6% by weight, including all ranges and subranges there-between, relative to the weight of the composition, of at least one silicone-organic polymer compound selected from Crotonic Acid/Vinyl C8-12 Isoalkyl Esters/VA/Bis-Vinyldimethicone Crosspolymer, at least one organic solvent, preferably in an amount of from about 2% to about 6% by weight, including all ranges and subranges there-between, relative to the weight of the composition, and preferably chosen from C2-C4 alcohols, glycols, glycerol, glycol ethers, and mixtures thereof, and water, wherein the weight ratio of the polyurethane latex polymer to the silicone-organic polymer compound is less than 2; and all weights on a dry weight basis.

In one embodiment, the composition of the present invention is a composition for styling hair and/or maintaining the style of hair.

In other embodiments, the cosmetically acceptable carrier comprises a mixture of water and a volatile organic solvent/compound chosen from C2 to C6 alcohols, glycols, glycerol, and mixtures thereof.

In one preferred embodiment, the composition of the present invention is in the form of a spray composition.

In other embodiments the composition of the present invention can contain a propellant.

In one embodiment, the composition of the present invention does not contain an anionic polymer other than the silicone-organic polymer compound of the present invention.

### Method of Making

The hair cosmetic composition of the invention is made by:

A. combining, in a cosmetically acceptable carrier,
(a) from about 0.25% to about 3.5% by weight of at least one polyurethane latex polymer; and
(b) from about 0.5% to about 6% by weight of at least one silicone-organic polymer compound;
   all weights being relative to the weight of the composition, and wherein the weight ratio of the polyurethane latex polymer to the silicone-organic polymer compound is less than 2; and
   B. mixing the ingredients in A in order to form the hair cosmetic composition.

A neutralizing agent may be employed in the above-described method in an amount sufficient to fully or partially neutralize, depending on the desired degree of neutralization, the at least one silicone-organic polymer compound.

In an embodiment, the at least one silicone-organic polymer compound is first combined with an amount of a neutralizing agent sufficient to fully or partially neutralize (as desired) the silicone-organic polymer compound. The fully or partially neutralized silicone-organic polymer compound is then combined and mixed with one or more of the rest of the ingredients of the hair cosmetic composition of the invention.

### Method of Use

The method or process of using the compositions of the present invention may depend on the type of hair being targeted and, consequently, on the specific ingredients contained in the composition used to style or shape or maintain the shape of hair.

An embodiment of the present invention is a method of styling or shaping or maintaining the shape of hair.

Another embodiment of the present invention is a method of controlling frizziness of hair comprising applying onto the hair, any one of the compositions of the present invention.

According to at least one embodiment, such a method comprises applying to the hair, an effective amount of any one of the compositions of the present invention.

The compositions of the present invention may be employed in an effective amount to adequately cover the surface of the fibers of the hair and to achieve a desirable or effective style or shape of the hair as well as a desirable degree of hold. The precise amount of composition to be applied onto the hair will thus depend on the degree of treatment/styling/shaping/hold desired.

An effective amount of the composition is typically from about 0.1 gram to about 50 grams, preferably from about 0.5 gram to about 20 grams of the composition. Application to the hair typically includes working the composition through the hair.

Further disclosed herein is the use of the compositions of the present invention for shaping or styling hair and/or retaining a hairstyle.

The compositions may be applied to wet or dry hair, before or after shaping. They may be used in a non-rinse fashion in order to impart or maintain the shape of the hair. In some other embodiments, the composition may be rinsed from the hair.

One embodiment of the present invention is a method of controlling frizziness of hair comprising contacting hair with the compositions of the invention, heating the hair and optionally, applying a smoothing action to the hair while heating the hair or after heating the hair or before heating the hair.

The hair that has been contacted with the compositions of the present invention may be air-dried and/or further styled or shaped by applying heat on the hair using a blow dryer, hood dryer or hair dryer, flat iron, heating implement, or other suitable devices, such as a hot iron or curling iron, and/or by combing or brushing or running the fingers through the hair. Other shaping tools may be chosen from combs and brushes.

In certain embodiments, the composition is allowed to remain (leave-on time) on the keratin fibers, for example, from about 1 to about 60 minutes, or such as from about 5 to about 45 minutes, or such as from about 5 to about 30 minutes, or such as from about 10 to about 20 minutes, or such as at about 20 minutes, or such as at about 10 minutes.

The smoothing action may be accomplished by use of suitable devices for brushing or smoothing the hair include a hair brush, comb, or flat iron. The smoothing action on the hair may also include running the fingers through the hair.

A suitable applicator device is an applicator brush.

Heat (at a temperature of at least 40° C.) can be applied to the hair while the smoothing action is performed on the hair. The heat source can be chosen from a blow dryer, a flat iron, a hair dryer, a heat lamp, a heat wand, or other similar devices.

In addition, independently of the embodiment use, the composition present on the fibers or hair is left in place for a time, generally, from about 1 to about 60 minutes, such as from about 5 to about 45 minutes, or such as from about 5 to about 20 minutes, or such as from about 10 to about 20 minutes, or such as of about 20 minutes or such as of about 10 minutes.

The compositions of the present invention are easy to spread on hair.

It has surprisingly and unexpectedly discovered that the application of the composition onto the hair results in satisfactorily controlling the frizziness of hair and/or making the shape or style of the hair humidity resistant

The frizz control and humidity resistant effects obtained using the compositions and methods of the present invention may also be durable or long-lasting, i.e., wash or shampoo resistant.

As used herein, “long-lasting” or “durable” frizz control or styling or shaping is understood to mean that the frizz control or humidity resistant or styling benefits imparted to hair by the compositions of the invention last over a period of time and/or over high humidity conditions (at least 60%) and/or after multiple wash cycles (with water or shampoo/water or shampoo/water/conditioner/water or conditioner/water). Said multiple wash cycles is understood to mean more than one wash cycle, such as two or three or four or five or six or seven or eight or nine or ten wash cycles.

The compositions of the present invention may be packaged in any suitable container such as a tube, a jar or a bottle. In certain embodiments, the composition can be packaged in a tube or bottle, for example, a squeeze tube or squeeze bottle.

Additionally, the applicator device can be attached or connected to the opening of the packaging tube or bottle wherein the applicator device is a brush or a comb with teeth such that the ends of the teeth have openings from which the composition of the invention can flow through and be applied directly onto the hair.

Another embodiment of the present invention is a method of controlling frizziness of hair comprising (a) providing the composition of the present invention, and (b) providing instructions for applying said composition to the hair.

Instructions for applying the composition of the present invention onto keratin fibers such as hair on the head or eyelashes may comprise directions of use of the composition for the end-user to follow. The end-user may be a consumer or cosmetologist or salon hair dresser. Directions may comprise instructing the end-user to take an amount of the composition in sufficient quantity such that the composition adequately covers the hair fibers and imparts the desired shape or style or hold to the hair fibers. Directions may additionally instruct the end-user to use a device such as a comb, brush (e.g., hair brush or brush wand), flat iron plates, blow dryer or the fingers for shaping or styling the hair or for separating the fibers of the hair. Directions may also additionally instruct the end-user to apply heat to the hair such as by blow drying the hair or using a heating device on the hair.

Instructions for applying the composition of the present invention onto keratin fibers such as hair may appear on the container (such as can, bottle or jar) holding the composition of the present invention or on the box or carton or other packaging comprising the container holding said composition.

It has been surprisingly and unexpectedly discovered that the combination of at least one polyurethane latex polymer, at least one silicone-organic polymer compound, an organic solvent, and water produced a composition that resulted in satisfactory styling or shaping of hair, including long lasting or durable style or wear on the hair, very good and durable/long lasting frizz control benefits, even when the hair contacted with the composition was exposed to high humidity conditions or subjected to multiple cycles of shampooing and/or washing with water.

The degree of styling or shaping hold on the hair may be evaluated by assessing the appearance of the hair, or quantitatively assessing the degree of frizziness or reduction in frizziness of the hair, or the retention of curl elongation or straightness of the hair after contacting the hair with the composition of the invention. The assessments can be continuously made over time, or with exposure to high humidity conditions or with multiple cycles of shampooing and/or washing with water.

As used herein, the method and composition disclosed herein may be used on the hair that has not been artificially dyed, pigmented or permed or on the hair that has been artificially dyed, pigmented or permed.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result.

### EXAMPLES

The following examples are intended to be non-restrictive and explanatory only, with the scope of the invention being defined by the claims.

The ingredient amounts/concentrations in the compositions/formulas described below are expressed in % by weight, relative to the total weight of the composition/formula.

### Procedure for Determination of Physical Properties of Latex Films

Film plating: The latex film was obtained by allowing a 30 gram water solution containing 4 grams of the latex polymer(s) to dry slowly in a 100 mL PFA Petri dish (100 mm diameter×15 mm height) at room temperature for at least 3 days.

Film measurement: The latex film, with known dimensions (length, width, thickness), was mounted on the Q800 Dynamic Mechanical Analysis from TA Instrument, and tested in a DMA Control Force mode. The stress/strain test was obtained using the following procedure:
Preload force: 0.001 N
Isothermal: 25° C.
Soak time: 0.5 minutes
Force ramp rate: 0.5N/min to 18 N

The test ended when the sample broke, 18 N force was reached, or maximum displacement was achieved (25.5 mm).

From the stress/strain curve, the Young's Modulus was calculated as the slope of the linear portion at about 0.01% Strain to about 1% Strain. From the stress/strain curve, the % Strain at the stress of 0.5 MPa was also reported.

A high Young's Modulus demonstrates a hard film, while a lower Young's Modulus represents a more elastic film. A high Strain demonstrates a stretchy, elastic film, while a lower Strain represents a more brittle film.

Film properties of latex polymers as measured according to the above-described “Procedure for Determination of Physical Properties of Latex Films”:

Acrylates copolymer, commercially available from BASF in an aqueous dispersion under the trade name of LUVIFLEX SOFT=Young's Modulus of 2758 MPa and strain, under stress at 0.5 MPa, of <0.01%.

Polyurethane-34, commercially available from BAYER in an aqueous dispersion under the trade name of BAYCUSAN C1001=Young's Modulus of 3 MPa and strain, under stress at 0.5 MPa, of 18.82%.

### Example 1

### Spray Formulas

Formula A, pH 6.05 (2% by weight* in total of acrylates copolymer and Polyurethane-34 and 4% by weight,* P1101^{a})

| Phase | Chemical Name | Trade Name | % wt |
|---|---|---|---|
| A1 | DI WATER | | 80.473 |
| A2 | PHENOXYETHANOL | PHENOXYETHANOL | 0.900 |
| | | (SEPPIC) | |
| A3 | ETHYLHEXYLGLYCERIN | | 0.100 |
| A4 | ACRYLATES COPOLYMER | LUVIFLEX SOFT | 5.560 |
| | (30%) DISPERSION | (BASF) | |
| | IN WATER | | |
| A5 | POLYURETHANE-34 | BAYCUSAN C 1001 | 1.560 |
| | (32 +/− 2%) | (BAYER MAETRIAL | |
| | DISPERSION IN WATER | SCIENCE) | |
| B1 | CROTONIC ACID/VINYL | BELSIL P1101^{a} | 8.000 |
| | C8-12 ISOALKYL | (WACKER) | |
| | ESTERS/VA/BIS- | | |
| | VINYLDIMETHICONE | | |
| | CROSSPOLYMER^{a} | | |
| | (approx. 50%) | | |
| | IN ALCOHOL | | |
| B2 | AMINOMETHYL | AMP ANGUS (DOW | 0.207 |
| | PROPANOL | CHEMICAL) | |
| B3 | DENATURED ALCOHOL | | 2.000 |
| C1 | PEG-40 HYDROGENATED | | 0.800 |
| | CASTOR OIL | | |
| C2 | FRAGRANCE | NIKIS (ROBERTET) | 0.400 |

* % dry weight basis

Formula B, pH 5.95 (2% by weight* acrylates copolymer and 4% by weight* P1101^{a})

| Phase | Chemical Name | Trade Name | % wt |
|---|---|---|---|
| A1 | DI WATER (QS) | | 80.184 |
| A2 | PHENOXYETHANOL | PHENOXYETHANOL | 0.900 |
| | | (SEPPIC) | |
| A3 | ETHYLHEXYLGLYCERIN | | 0.100 |
| A4 | ACRYLATES COPOLYMER | LUVIFLEX SOFT | 7.400 |
| | (30%) DISPERSION | (BASF) | |
| | IN WATER | | |
| B1 | CROTONIC ACID/VINYL | BELSIL P1101^{a} | 8.000 |
| | C8-12 ISOALKYL | (WACKER) | |
| | ESTERSA/A/BIS- | | |
| | VINYLDIMETHICONE | | |
| | CROSSPOLYMER^{a} | | |
| | (approx. 50%) | | |
| | IN ALCOHOL | | |
| B2 | AMINOMETHYL | ANGUS (DOW | 0.216 |
| | PROPANOL | CHEMICAL) | |
| B3 | DENATURED ALCOHOL | | 2.000 |
| C1 | PEG-40 HYDROGENATED | | 0.800 |
| | CASTOR OIL | | |
| C2 | FRAGRANCE | NIKIS (ROBERTET) | 0.400 |

* % dry weight basis

Inventive Formula C, pH 8.58 (2% by weight* Polyurethane-34 and 4% by weight* P1101^{a})

| Phase | Chemical Name | Trade Name | % wt |
|---|---|---|---|
| A1 | DI WATER (QS) | | 81.334 |
| A2 | PHENOXYETHANOL | PHENOXYETHANOL | 0.900 |
| | | (SEPPIC) | |
| A3 | ETHYLHEXYLGLYCERIN | | 0.100 |
| A4 | POLYURETHANE-34 | BAYCUSAN C 1001 | 6.250 |
| | (32 +/− 2%) | (BAYER) | |
| | DISPERSION IN WATER | | |
| B1 | CROTONIC ACID/VINYL | BELSIL P1101^{a} | 8.000 |
| | C8-12 ISOALKYL | (WACKER) | |
| | ESTERS/VA/BIS- | | |
| | VINYLDIMETHICONE | | |
| | CROSSPOLYMER^{a} | | |
| | (approx. 50%) | | |
| | IN ALCOHOL | | |
| B2 | AMINOMETHYL | ANGUS (DOW | 0.216 |
| | PROPANOL | CHEMICAL) | |
| B3 | DENATURED ALCOHOL | | 2.000 |
| C1 | PEG-40 HYDROGENATED | | 0.800 |
| | CASTOR OIL | | |
| C2 | FRAGRANCE | NIKIS (ROBERTET) | 0.400 |

* % dry weight basis

Formula D, pH 8.73 (4% by weight* P1101^{a})

| Phase | Chemical Name | Trade Name | % wt |
|---|---|---|---|
| A1 | DI WATER (QS) | | 87.584 |
| A2 | PHENOXYETHANOL | PHENOXYETHANOL | 0.900 |
| | | (SEPPIC) | |
| A3 | ETHYLHEXYLGLYCERIN | | 0.100 |
| B1 | CROTONIC ACID/VINYL | BELSIL P1101^{a} | 8.000 |
| | C8-12 ISOALKYL | (WACKER) | |
| | ESTERS/VA/BIS- | | |
| | VINYLDIMETHICONE | | |
| | CROSSPOLYMER^{a} | | |
| | (approx. 50%) IN | | |
| | ALCOHOL | | |
| B2 | AMINOMETHYL | ANGUS (DOW | 0.216 |
| | PROPANOL | CHEMICAL) | |
| B3 | DENATURED ALCOHOL | | 2.000 |
| C1 | PEG-40 HYDROGENATED | | 0.800 |
| | CASTOR OIL | | |
| C2 | FRAGRANCE | NIKIS (ROBERTET) | 0.400 |

* % dry weight basis

### Example 2

### Spray Formulas (Varying Amounts of Polyurethane-34 and P1101)

| | | Formula 1 | Formula 2 | Formula 3 | Formula 4 | Formula 5 |
|---|---|---|---|---|---|---|
| | | (2, 4)* | (1, 2)* | (1, 2.5)* | (2, 2.5)* | (2, 2)* |
| | | pH 8.96 | pH 8.76 | pH 8.77 | pH 8.75 | pH 8.85 |
| Phase | Chemical Name | % wt | % wt | % wt | % wt | % wt |
| A1 | DI WATER (QS) | 81.343 | 88.572 | 87.546 | 84.421 | 85.447 |
| A2 | PHENOXYETHANOL | 0.900 | 0.900 | 0.900 | 0.900 | 0.900 |
| | (SEPPIC) | | | | | |
| A3 | ETHYLHEXYLGLYCERIN | 0.100 | 0.100 | 0.100 | 0.100 | 0.100 |
| A4 | POLYURETHANE-34 | 6.250 | 3.125 | 3.125 | 6.250 | 6.250 |
| | (32 +/− 2%) DISPERSION | | | | | |
| | IN WATER | | | | | |
| | BAYCUSAN C 1001 | | | | | |
| | (BAYER) | | | | | |
| B1 | CROTONIC ACID/VINYL | 8.000 | 4.000 | 5.000 | 5.000 | 4.000 |
| | C8-12 ISOALKYL | | | | | |
| | ESTERS/VA/BIS- | | | | | |
| | VINYLDIMETHICONE | | | | | |
| | CROSSPOLYMER^{a} | | | | | |
| | (approx. 50%) IN | | | | | |
| | ALCOHOL | | | | | |
| | BELSIL P1101 (WACKER) | | | | | |
| B2 | AMINOMETHYL | 0.207 | 0.103 | 0.129 | 0.129 | 0.103 |
| | PROPANOL [ANGUS | | | | | |
| | (DOW CHEMICAL)] | | | | | |
| B3 | DENATURED ALCOHOL | 2.000 | 2.000 | 2.000 | 2.000 | 2.000 |
| C1 | PEG-40 | 0.800 | 0.800 | 0.800 | 0.800 | 0.800 |
| | HYDROGENATED | | | | | |
| | CASTOR OIL | | | | | |
| C2 | FRAGRANCE | 0.400 | 0.400 | 0.400 | 0.400 | 0.400 |

*% dry weight basis

* % dry weight basis

### Process of Making the Formulas:

The formulas above consist of three phases; A, B, and C. In phase A, all raw materials were added separately and mixed until uniform. In phase B (separate beaker), the respective raw materials were mixed until the solution was uniform, indicating that Crotonic acid/vinyl C8-12 isoalkyl esters/VA/bis-vinyldimethicone crosspolymer is neutralized to 100%. This phase was then added to the main tank. In phase C (separate beaker), Peg-40 Hydrogenated castor oil was first melted at 40-45° C., and followed by the addition of the fragrance. These raw materials were then mixed until uniformity, followed by the addition of this phase to the main tank. The raw materials were added in order of representation on the respective charts. Rayneri mixers were used to batch all of the formulations.

Note: Crotonic acid/vinyl C8-12 isoalkyl esters/VA/bis-vinyldimethicone crosspolymer was neutralized to 100%, based on the acid value of the lot number of the resin, in all formulas. Also, P1101 levels of up to 8% by wt (4% active) were used in the examples to test for performance efficacy on hair swatches, while P1101 levels of up to 5% by wt (2.5% active), which were the preferred in vivo levels, were used on hair of human heads. The maximum concentration of polyurethane-34 used in the inventive formulas was 2% by wt, on a dry weight basis, to prevent white flakes from forming on the hair.

### Example 3

### Testing for Shampoo/Wash-Resistant Frizz Control

A. The formulas in Example 1 above were tested on curly/frizzy hair swatches. Test Procedure using width (frizz control) measurements on swatches:
1. Hair swatches, 1.5+/−0.10 g each of curly/frizzy hair were prepared; 2 swatches for each treatment and 2 swatches for control 511 S (20 swatches total).
2. The hair swatches were washed with a cleansing cream by thoroughly wetting each swatch, applying 0.5 g of shampoo to the hair, spreading it along the hair until saturation (up to 30 sec), then washing it off under running water (up to 30 sec) at 35° C.+/−5° C.
3. The swatches were air dried overnight at ambient temperature. Each swatch was then combed through 3 times. The width of each hair swatch was measured (T₀). The width was measured at the ends, including fly-aways.
4. 0.20 g+/−0.05 g of the test formula(s) was applied onto each swatch. Each swatch was then combed through 3 times, and the test formulas were allowed to saturate the hair for 10 minutes.
5. For heat treatment: the swatches were flat-ironed at 1″/sec, 450° F., 3 passes each and the next step was conducted. For no heat treatment the next step was conducted.
6. Once the swatches were dry, each swatch was combed through 3 times. Second width measurements were taken (T₁).
7. Swatches were then washed with cleansing cream (0.5 g) and left to air dry at ambient temperature. Dried swatches were combed through 3 times each. Third width measurements were taken (T₂). This measurement was taken after the first wash cycle.
8. Step 7 (wash cycle) was repeated and width measurements were taken (T₃). If (T₀−T₃)/T₀≦0.05 (i.e., swatch returned to initial width), then the wash cycle was stopped.
9. Otherwise, the wash cycles and width measurements were continued (T₄, T₅, etc.) until (T₀−T_{N})/T₀≦0.05.

### Spray Compositions Tested on Hair Swatches:

TABLE 1
Air dry: Width measurements

| | | Width (cm) at Time Period (T) | | | | | | | | | | |
|---|---|---|---|---|---|---|---|---|---|---|---|---|
| Fla | #* | (T₀) | (T₁) | (T₂) | (T₃) | (T₄) | (T₅) | (T₆) | (T₇) | (T₈) | (T₉) | (T₁₀) |
| Water | 1 | 4.0 | 7.2 | 8.0 | 5.4 | 4.9 | 3.3 | 3.5 | 3.7 | 4.5 | 5.0 | 4.7 |
| (control) | 2 | 4.9 | 8.3 | 5.5 | 5.0 | 5.1 | 4.5 | 4.3 | 3.4 | 4.0 | 4.0 | 5.5 |
| A | 3 | 7.5 | 6.0 | 5.0 | 4.2 | 4.8 | 4.5 | 4.5 | 4.1 | 4.5 | 5.4 | 5.8 |
| | 4 | 4.2 | 4.3 | 4.5 | 4.2 | 4.2 | 3.3 | 3.5 | 3.5 | 4.5 | 4.8 | 5.5 |
| B | 5 | 5.1 | 5.5 | 3.8 | 4.4 | 5.3 | 4.4 | 4.5 | 5.8 | 5.1 | 5.3 | 4.9 |
| | 6 | 6.0 | 6.5 | 3.7 | 4.0 | 3.3 | 4.5 | 4.2 | 4.2 | 4.3 | 4.7 | 5.0 |
| C | 7 | 6.8 | 5.4 | 5.4 | 3.8 | 3.2 | 4.0 | 4.7 | 5.5 | 4.2 | 4.4 | 5.8 |
| | 8 | 6.4 | 5.4 | 3.0 | 1.3 | 2.8 | 3.8 | 3.5 | 3.6 | 4.3 | 5.0 | 5.0 |
| D | 9 | 6.0 | 4.3 | 3.4 | 4.0 | 4.4 | 4.5 | 3.5 | 4.0 | 4.0 | 4.1 | 4.2 |
| | 10 | 5.5 | 2.9 | 4.3 | 3.5 | 3.2 | 3.5 | 3.1 | 3.7 | 4.5 | 5.5 | 5.5 |

*Swatch number

TABLE 2
With Heat, Flat-iron (450° F.): Width measurements

| | | Width (cm) at Time Period (T) | | | | | | | | | | |
|---|---|---|---|---|---|---|---|---|---|---|---|---|
| Fla | #* | (T₀) | (T₁) | (T₂) | (T₃) | (T₄) | (T₅) | (T₆) | (T₇) | (T₈) | (T₉) | (T₁₀) |
| Water | 11 | 8.0 | 3.8 | 6.0 | 4.5 | 5.0 | 4.0 | 5.0 | 4.3 | 3.3 | 4.2 | 4.8 |
| (control) | 12 | 7.0 | 5.6 | 5.6 | 4.7 | 4.9 | 3.4 | 3.5 | 4.4 | 4.7 | 3.5 | 4.1 |
| A | 13 | 8.5 | 2.9 | 3.5 | 1.9 | 2.0 | 3.2 | 3.3 | 3.5 | 4.7 | 4.5 | 4.5 |
| | 14 | 5.2 | 2.7 | 3.0 | 1.8 | 2.5 | 2.5 | 3.7 | 3.2 | 2.8 | 3.1 | 4.0 |
| B | 15 | 6.5 | 3.5 | 2.7 | 3.7 | 4.5 | 4.1 | 4.8 | 4.1 | 3.8 | 5.0 | 4.2 |
| | 16 | 6.2 | 2.7 | 2.2 | 3.0 | 3.6 | 3.5 | 4.4 | 3.3 | 4.0 | 3.2 | 4.0 |
| C | 17 | 5.8 | 2.1 | 2.2 | 2.0 | 1.5 | 2.0 | 2.2 | 1.5 | 2.3 | 2.3 | 2.4 |
| | 18 | 6.7 | 3.5 | 2.6 | 2.5 | 2.2 | 2.6 | 2.5 | 1.8 | 2.6 | 3.1 | 3.5 |
| D | 19 | 5.3 | 3.3 | 2.2 | 2.0 | 3.2 | 2.9 | 4.6 | 3.0 | 2.9 | 3.7 | 3.0 |
| | 20 | 8.5 | 3.8 | 2.7 | 2.9 | 2.2 | 3.4 | 3.3 | 3.1 | 3.5 | 3.8 | 4.0 |

*Swatch number

Based on the average of the width measurements on two swatches per time period, Formula C imparted the greatest degree of frizz control on the treated swatches, with frizz control being achieved for at least 9 wash cycles (T10) when the hair was flat-ironed. The swatches treated with the comparative Formula A, D, and B provided frizz control up to T8 (7 wash cycles), T8 (7 wash cycles), and T4 (3 wash cycles), respectively. Additionally, under the air-drying conditions, swatches treated with the inventive formula showed the greatest width reduction for up to three wash cycles (T4).

Shampoo/wash-resistant frizz control as provided by the inventive formula C indicate durable and/or long lasting and/or long-term styling or shaping effects as well as durable and/or long lasting frizz control provided by a shampoo/wash-resistant film or composition on the hair.

Furthermore, after formula application, the hair treated with Formulas A and B felt stiffer compared to the hair treated with the inventive formula C, especially when the hair was heat-treated or flat-ironed.

Length measurements were also made on the swatches at the different time points in order to assess a curl elongation effect based on the reversion of the elongated or straightened hair back to initial curl length. Thus, the smaller the change in length of the swatch over time compared to the length immediately upon treating the swatch, the more the swatch was able to remain straightened or elongated. Based on these measurements, the change in length over time among the swatches treated with the inventive formula was comparable for both air-dried and flat-ironed swatches. Overall, the effect of shampoo/wash-resistant frizz control (width reduction) was observed over a greater number of wash cycles compared to shampoo/wash-resistant curl elongation (length). Therefore, the findings of more significant frizz control compared to curl elongation effects suggests that the association of PU-34 and P1101 results in hair frizz control through fiber alignment without distortion of the disulfide bonds within the keratinous fibers.

B. The formulas in Example 2 above were tested on curly/frizzy hair swatches in high humidity conditions (80%).

### Test Procedure:

1. Swatches of curly/frizzy hair were prepared (1.5+/−0.10 g each); 2 swatches for each treatment and 2 swatches for control 511 S (24 swatches total).
2. Hair swatches were washed with cleansing cream by thoroughly wetting each swatch, applying 1 g of shampoo to hair, spreading shampoo along the hair swatch until saturation (up to 30 sec), then washing the shampoo off under running water (up to 30 sec). The water temperature was at 35° C.+/−5° C.
3. Swatches were air-dried overnight at room temperature. Each swatch was combed through 3 times. The width of each hair swatch was measured (T₀). The width was measured at the ends, including fly-aways.
4. 0.15 g+/−0.05 g of the test formula(s) was applied onto each swatch. Each swatch was then combed through 3 times, and the formulas were allowed to saturate the hair for 10 minutes.
5. For heat treatment: the swatches were flat-ironed at r/sec, at 450° F., 3 passes each. For no-heat treatment: proceed to next step.
6. Once swatches were dry, they were combed through 3 times. Second width measurements were taken (T₁).
7. Swatches were placed in the humidity chamber at controlled humidity (80% RH) for 16 hrs. Width measurements were taken (T₂).
8. Swatches were then washed with cleansing cream (1 g), and left to air dry at ambient temperature. Once dried, the swatches were brushed 3 times, and then were again washed with cleansing cream, and air dried. The dried swatches were brushed 3 times and then placed in a humidity chamber at controlled humidity (80% RH) for 16 hrs. Width measurements were taken (T4)(after the second wash cycle).
9. Step 8 was repeated until T_{1/2} was taken (10 washes total) or until (T₀−T_{N})/T₀≦0.05 (i.e., return to initial width).

### Spray Compositions Tested on Hair Swatches:

TABLE 3
Air dry: Width measurements

Width (cm) at Time Period (T)

| Fla | #* | (T₀) | (T₁) | (T₂) | (T₄) | (T₆) |
|---|---|---|---|---|---|---|
| Water | 13 | 9.7 | 10.5 | 10.8 | 9.4 | 9.5 |
| (control) | 14 | 10.6 | 11.0 | 11.4 | 9.8 | 10.8 |
| 1 | 15 | 11.0 | 7.5 | 6.9 | 7.0 | 6.9 |
| | 16 | 11.5 | 9.0 | 8.4 | 7.5 | 7.1 |
| 2 | 17 | 10.5 | 9.5 | 8.8 | 7.6 | 6.8 |
| | 18 | 7.0 | 6.7 | 4.6 | 7.3 | 7.5 |
| 3 | 19 | 12.0 | 7.7 | 7.6 | 7.1 | 7.2 |
| | 20 | 10.0 | 7.5 | 7.6 | 7.4 | 8.5 |
| 4 | 21 | 8.1 | 8.4 | 7.7 | 7.6 | 7.3 |
| | 22 | 7.6 | 6.8 | 6.9 | 8.0 | 8.3 |
| 5 | 23 | 9.0 | 9.0 | 7.4 | 8.2 | 7.4 |
| | 24 | 8.0 | 8.4 | 9.4 | 6.8 | 8.5 |

*Swatch number

TABLE 4
With Heat, Flat-iron (450° F.): Width measurements

Width (cm) at Time Period (T)

| Fla | #* | (T₀) | (T₁) | (T₂) | T₄) | (T₆) |
|---|---|---|---|---|---|---|
| Water | 1 | 9.7 | 5.0 | 11.2 | 9.7 | 10.5 |
| (control) | 2 | 11.2 | 6.4 | 7.0 | 8.8 | 7.9 |
| 1 | 3 | 10.7 | 6.4 | 7.0 | 7.3 | 8.3 |
| | 4 | 10.4 | 5.0 | 7.0 | 7.4 | 6.5 |
| 2 | 5 | 10.6 | 5.0 | 6.1 | 7.0 | 5.4 |
| | 6 | 7.0 | 4.3 | 6.7 | 8.4 | 6.2 |
| 3 | 7 | 9.5 | 5.5 | 7.4 | 6.0 | 6.4 |
| | 8 | 11.5 | 6.5 | 6.4 | 7.2 | 7.5 |
| 4 | 9 | 9.5 | 5.2 | 7.9 | 6.6 | 7.6 |
| | 10 | 11.5 | 5.2 | 6.2 | 9.4 | 7.4 |
| 5 | 11 | 11.9 | 8.5 | 7.5 | 7.4 | 5.2 |
| | 12 | 11.4 | 5.5 | 5.5 | 6.5 | 6.0 |

*Swatch number

Based on the average of the measurements on two swatches per time period, the swatches treated with different ratios of PU-34 and P1101 showed shampoo/wash-resistant frizz control under high humidity conditions (80%), indicating durable and/or long lasting or long-term styling benefits imparted by a shampoo/wash-resistant film or composition on the hair. In particular, application of PU-34/P1101 to the hair swatches (air dried and flat ironed) resulted in significant width reduction even after 4 wash cycles (T6).

Length measurements were also made on the swatches at the different time points in order to assess the curl elongation effect (described above). Based on these measurements, the change in length over time among the treated swatches was comparable for both air-dried and flat-ironed swatches. Overall, the effect of shampoo/wash-resistant frizz control (width reduction) was observed over a greater number of wash cycles compared to shampoo/wash-resistant curl elongation (length). Therefore, the findings of more significant frizz control compared to curl elongation effects suggests that the association of PU-34 and P1101 results in hair frizz control through fiber alignment without distortion of the disulfide bonds within the keratinous fibers.

The hair swatches treated with the inventive formula of 1% active PU-34+2.5% active P1101 felt the softest. On a curly/frizzy haired mannequin head, this formula imparted shine, shapeability, smoothness, and a flexible hold to the hair. In a subsequent test wherein swatches treated with the inventive formula were compared to swatches treated with PU-34 alone, P1101 alone, and PU-34+PEG/PPG-17/18 dimethicone (comparative formulas), the inventive formula was found to provide the greatest degree of frizz control at high humidity conditions (80%, 16 hour exposure) under air-drying and flat-ironing conditions (before washing the hair). This frizz control benefit from the inventive formula was more noticeable in the heat treated (flat-ironed) swatches. After two and four cycles of shampooing/washing and exposure to 80% humidity for 16 hours, the inventive formula performed comparably to the comparative formulas with respect to frizz control. However, with respect to cosmeticity and sensorial benefits to hair, the inventive formula provided smoothness, a good cosmetic feel, and desirable shine to hair and ease of brushing the hair even after the multiple cycles of shampoo/wash/humidity exposure. In contrast, compared to the hair treated with the inventive formula, the hair treated with PU-34 alone gave some resistance during brushing and had less shine, and the hair treated with the PU-34/PEG/PPG-17/18 dimethicone combination felt dry to the touch after drying the hair and had less shine.

The foregoing description illustrates and describes the present invention. Additionally, the invention shows and describes only the preferred embodiments of the invention, but, as mentioned above, it is to be understood that it is capable of changes or modifications within the scope of the concept as expressed herein, commensurate with the above teachings and/or skill or knowledge of the relevant art. The embodiments described hereinabove are further intended to explain best modes known of practicing the invention and to enable others skilled in the art to utilize the invention in such, or other, embodiments and with the various modification required by the particular applications or uses disclosed herein. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicated to be incorporated by reference. In the case of inconsistencies, the present invention will prevail.

What is claimed is:

1. A hair cosmetic composition comprising, in a cosmetically acceptable carrier:
(a) from about 0.25% to about 3.5% by weight of at least one polyurethane latex polymer; and
(b) from about 0.5% to about 6% by weight of at least one silicone-organic polymer compound;
all weights being relative to the weight of the composition, and wherein the weight ratio of the polyurethane latex polymer to the silicone-organic polymer compound is less than 2.

2. The hair cosmetic composition of claim 1, wherein the at least one polyurethane latex polymer is a film-forming polymer and is dispersed as particles in an aqueous dispersion medium.

3. The hair cosmetic composition of claim 2, wherein the at least one polyurethane latex polymer is selected from a polyurethane latex polymer having a Young's modulus ranging from about 0.1 MPa to about 10 MPa, and a strain, under stress at 0.5 MPa, of at least about 1%, a polyurethane latex polymer having a having a Young's modulus ranging from about 10 MPa to about 6 GPa, and a strain, under stress at 0.5 MPa, of less than about 5%, and mixtures thereof.

4. The hair cosmetic composition of claim 2, wherein the at least one latex polyurethane polymer is selected from polyurethane-34.

5. The hair cosmetic composition of claim 4, wherein polyurethane-34 has a Young's modulus ranging from about 0.1 MPa to about 10 MPa, and a strain, under stress at 0.5 MPa, of at least about 1%.

6. The hair cosmetic composition of claim 4, wherein the at least one latex polyurethane polymer is present in an amount ranging from about 0.375% to about 2.55% by weight, relative to the total weight of the composition.

7. The hair cosmetic composition of claim 6, wherein the at least one polyurethane latex polymer is present in an amount ranging from about 0.45% to about 2.2% by weight, relative to the total weight of the composition.

8. The hair cosmetic composition of claim 4, wherein the silicone-organic polymer compound is dispersed in an alcoholic medium.

9. The hair cosmetic composition of claim 8, wherein the silicone-organic polymer compound is neutralized by a neutralizing agent selected from aminomethyl propanol, aminomethyl propanediol, triisopropanol amine) sodium hydroxide, potassium hydroxide, dimethylstearylamine, dimethyl/tallowamine lysine, ornithine, arginine, monoethanolamine, triethanolamine, calcium hydroxide, calcium bicarbonate, and mixtures thereof.

10. The hair cosmetic composition of claim 9, wherein the silicone-organic polymer compound is Crotonic Acid/Vinyl C8-12 Isoalkyl Esters/VA/Bis-Vinyldimethicone Crosspolymer.

11. The hair cosmetic composition of claim 8, wherein the silicone-organic polymer compound is present in an amount of from about 1% to about 5% by weight, relative to the total weight of the composition.

12. The hair cosmetic composition of claim 11, wherein the silicone-organic polymer compound is present in an amount of from about 2% to about 4% by weight, relative to the total weight of the composition.

13. The hair cosmetic composition of claim 12, wherein the weight ratio of the polyurethane latex polymer to the silicone-organic polymer compound is from about 0.1 to about 1.75.

14. The hair cosmetic composition of claim 13, wherein the weight ratio of the polyurethane latex polymer to the silicone-organic polymer compound is from about 0.25 to about 1.1.

15. The hair cosmetic composition of claim 13, wherein the cosmetically acceptable carrier comprises water.

16. The hair cosmetic composition of claim 15, wherein the cosmetically acceptable carrier further comprises at least one organic solvent is present in an amount of from about 0.5% to about 60% by weight, relative to the total weight of the composition.

17. The hair cosmetic composition of claim 16, wherein the at least one organic solvent is selected from ethanol, isopropyl alcohol, butanol, propylene glycol, butylene glycol, pentylene glycol, hexylene glycol, glycerol, isododecane, acetone, propylene carbonate, benzyl alcohol, mineral oil, polybutene, hydrogenated polyisobutene, hydrogenated polydecene, polydecene, squalene, petrolatum, ethylene glycol monomethyl ether, ethylene glycol monopropyl ether, ethylene glycol monobutyl ether, propylene glycol monomethyl ether, propylene glycol monopropyl ether, propylene glycol monobutyl ether, diethylene glycol monoethyl ether, diethylene glycol monobutyl ether, dipropylene glycol n-butyl ether, and mixtures thereof.

18. The hair cosmetic composition of claim 17, further comprising at least one auxiliary ingredient selected propellants, rheology modifiers, surfactants, lipohilic compounds, skin and hair active agents, sunscreens, preservatives, fragrances, pH adjusting agents, and mixtures thereof.

19. The hair cosmetic composition of claim 17, further comprising a nonionic surfactant selected from fatty alcohols, alkoxylated alcohols, glyceryl esters, alkylpolyglycosides, and mixtures thereof.

20. The hair cosmetic composition of claim 18, further comprising a propellant.

21. The hair cosmetic composition of claim 20, wherein the composition is in the form of a spray.

22. The hair cosmetic composition of claim 1, wherein the hair cosmetic composition is a hair styling or hair shaping composition.

23. A method of imparting durable styling or shaping properties to hair and/or of controlling frizziness of hair, the method comprising: (a) providing the hair cosmetic composition of claim 1, and (b) providing instructions on applying the hair cosmetic composition to hair.

24. A hair cosmetic composition for controlling frizziness of hair, the hair cosmetic composition comprising:
(a) from about 0.375% to about 2.55% by weight of at least one polyurethane latex polymer selected from polyurethane-34;
(b) from about 1% to about 5% by weight of at least one silicone-organic polymer compound selected from Crotonic Acid/Vinyl C8-12 Isoalkyl Esters/VA/Bis-Vinyldimethicone Crosspolymer;
(c) from about 1.5% to about 8% by weight of at least one organic solvent;
(d) water; and
wherein the weight ratio of the polyurethane latex polymer to the silicone-organic polymer compound is from about 0.1 to about 1.5.

25. A method of imparting durable styling or shaping properties to hair and/or of controlling frizziness of hair, the method comprising:
(1) contacting hair with a hair cosmetic composition comprising, in a cosmetically acceptable carrier:
(a) from about 0.25% to about 3.5% by weight of at least one polyurethane latex polymer; and
(b) from about 0.5% to about 6% by weight of at least one silicone-organic polymer compound;
all weights being relative to the weight of the composition;
wherein the weight ratio of the polyurethane latex polymer to the silicone-organic polymer compound is less than 2;
wherein the cosmetically acceptable carrier comprises water and from about 0.5% to about 60% by weight at least one organic solvent; and
(2) optionally, heating the hair; and
(3) optionally, applying a smoothing action to the hair while heating the hair or after heating the hair or before heating the hair.

26. The method according to claim 19, further comprising a step of treating the hair with heat at a temperature ranging from about 25° C. to about 250° C. before, during, or after the application of the hair cosmetic composition.
